(19) United States
(12) Patent Application Publication
(10) Pub. No.: US 2012/0208800 A1
(43) Pub. Date: Aug. 16, 2012

(54) Bromodomain Inhibitors For Treating Autoimmune And Inflammatory Diseases

(76) Inventors: Chun-Wa Chung, Stevenage (GB); Edwige Nicodeme, Les Ulis (FR)

(21) Appl. No.: 13/501,345

(22) PCT Filed: Nov. 3, 2010
(86) PCT No.: PCT/EP2010/066695

§ 371 Date: Apr. 11, 2012

(30) Foreign Application Priority Data
Nov. 5, 2009 (GB) 0919432.5

(51) Int. Cl. A61K 31/4706; A61P 29/00; A61P 37/06; A61K 31/5517; A61K 31/4745

(52) U.S. Cl. 514/220; 514/313; 514/293

(57) ABSTRACT

The use of compounds in the treatment of autoimmune and inflammatory diseases or conditions, pharmaceutical compositions containing such compounds and to methods for identifying compounds for use in the treatment of such diseases or conditions.

### FIELD OF THE INVENTION

The present invention relates to the use of compounds in the treatment of autoimmune and inflammatory diseases or conditions, to pharmaceutical compositions containing such compounds and to methods for identifying compounds for use in the treatment of such diseases or conditions.

### BACKGROUND OF THE INVENTION

The genomes of eukaryotic organisms are highly organised within the nucleus of the cell. The long strands of duplex DNA are wrapped around an octomer of histone proteins (most usually comprising two copies of histones H2A, H2B H3 and H4) to form a nucleosome. This basic unit is then further compressed by the aggregation and folding of nucleosomes to form a highly condensed chromatin structure. A range of different states of condensation are possible, and the tightness of this structure varies during the cell cycle, being most compact during the process of cell division. Chromatin structure plays a critical role in regulating gene transcription, which cannot occur efficiently from highly condensed chromatin. The chromatin structure is controlled by a series of post translational modifications to histone proteins, notably histones H3 and H4, and most commonly within the histone tails which extend beyond the core nucleosome structure. These modifications include acetylation, methylation, phosphorylation, ubiquitinylation, SUMOylation. These epigenetic marks are written and erased by specific enzymes, which place the tags on specific residues within the histone tail, thereby forming an epigenetic code, which is then interpreted by the cell to allow gene specific regulation of chromatin structure and thereby transcription.

Histone acetylation is most usually associated with the activation of gene transcription, as the modification loosens the interaction of the DNA and the histone octomer by changing the electrostatics. In addition to this physical change, specific proteins bind to acetylated lysine residues within histones to read the epigenetic code. Bromodomains are small (˜110 amino acid) distinct domains within proteins that bind to acetylated lysine resides commonly but not exclusively in the context of histones. There is a family of around 50 proteins known to contain bromodomains, and they have a range of functions within the cell.

The BET family of bromodomain containing proteins comprises 4 proteins (BRD2, BRD3, BRD4 and BRD-t) which contain tandem bromodomains capable of binding to two acetylated lysine residues in close proximity, increasing the specificity of the interaction. BRD2 and BRD3 are reported to associate with histones along actively transcribed genes and may be involved in facilitating transcriptional elongation (Leroy et al, Mol. Cell. 2008 30(1):51-60), while BRD4 appears to be involved in the recruitment of the pTEF-β complex to inducible genes, resulting in phosphorylation of RNA polymerase and increased transcriptional output (Hargreaves et al, Cell, 2009 138(1): 129-145). BRD-t is uniquely expressed in the testes and ovary. All family members have been reported to have some function in controlling or executing aspects of the cell cycle, and have been shown to remain in complex with chromosomes during cell division—suggesting a role in the maintenance of epigenetic memory. In addition some viruses make use of these proteins to tether their genomes to the host cell chromatin, as part of the process of viral replication (You et al Cell, 2004 117(3):349-60).

Japanese patent application JP2008-156311 discloses a benzimidazole derivative which is said to be a BRD2 bromodomain binding agent which has utility with respect to virus infection/proliferation.

Patent application WO2009/084693 discloses a series of thienotriazolodiazepiene derivatives that are said to inhibit the binding between an acetylated histone and a bromodomain containing protein which are said to be useful as anti-cancer agents.

It has now been found that compounds which inhibit the binding of a bromodomain with its cognate acetylated proteins have utility in the treatment of a range of autoimmune and inflammatory diseases or conditions.

### SUMMARY OF THE INVENTION

In a first aspect of the present invention, there is provided a method of treating autoimmune and inflammatory diseases or conditions which comprises administering to a subject in need thereof a therapeutically effective amount of a bromodomain inhibitor.

In a second aspect of the present invention, there is provided a bromodomain inhibitor for use in the treatment of autoimmune and inflammatory diseases or conditions.

In a third aspect of the present invention, there is provided the use of a bromodomain inhibitor in the manufacture of a medicament for the treatment of autoimmune and inflammatory diseases or conditions.

In a fourth aspect the present invention provides a pharmaceutical formulation comprising a bromodomain inhibitor and at least one pharmaceutical carrier, diluent or excipient, wherein the bromodomain inhibitor is present in an amount effective for use in the treatment of autoimmune and inflammatory diseases or conditions.

In a fifth aspect the present invention provides a method for identifying compounds for use in treating autoimmune and inflammatory diseases or conditions which comprises the step of determining whether the compound inhibits the binding of a bromodomain with its cognate acetylated protein.

### DETAILED DESCRIPTION OF THE INVENTION

The present invention provides for a method of treating autoimmune and inflammatory diseases or conditions which comprises administering to a subject in need thereof a therapeutically effective amount of a bromodomain inhibitor.

Suitably, the subject in need thereof is a mammal, particularly a human.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “bromodomain inhibitor” denotes a compound which inhibits the binding of a bromodomain with its cognate acetylated proteins. In one embodiment the bromodomain inhibitor is a compound which inhibits the binding of a bromodomain to acetylated lysine residues. In a further embodiment the bromodomain inhibitor is a compound which inhibits the binding of a bromodomain to acetylated lysine residues on histones, particularly histones H3 and H4.

In a particular embodiment the bromodomain inhibitor is a compound that inhibits the binding of BET family bromodomains to acetylated lysine residues (hereafter referred to as a “BET family bromodomain inhibitor”). In one embodiment the BET family bromodomain is BRD2, BRD3 or BRD4, in particular BRD2 or BRD3. A BET family bromodomain inhibitor is a compound which has a pIC₅₀≧5.0 of at least in one or more of the binding assays described herein.

In one embodiment the bromodomain inhibitor is a compound being a small molecule, in particular having a molecular weight of less 750, more particularly less than 500.

In one embodiment the bromodomain inhibitor is a compound selected from the group consisting of Examples 1-6 as shown in Table 1.

TABLE 1

| | Name | Structure |
|---|---|---|
| Example 1 | 1-methylethyl ((2S,4R)-1-acetyl-2- methyl-6-{4- [(methylamino)methyl]phenyl}-1,2,3,4- tetrahydro-4-quinolinyl)carbamate | 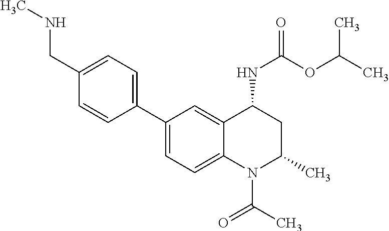 |
| Example 2 | 2-[(4S)-6-(4-Chlorophenyl)-1-methyl-8- (methyloxy)-4H-[1,2,4]triazolo[4,3- a][1,4]benzodiazepin-4-yl]-N- ethylacetamide | 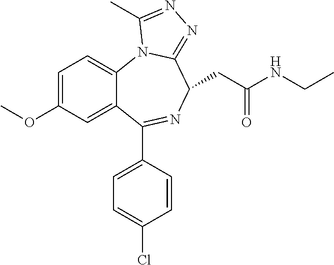 |
| Example 3 | 7-(3,5-dimethyl-4-isoxazolyl)-8- (methoxy)-1-[(1R)-1-(2-pyridinyl)ethyl]- 1,3-dihydro-2H-imidazo[4,5-c]quinolin- 2-one | 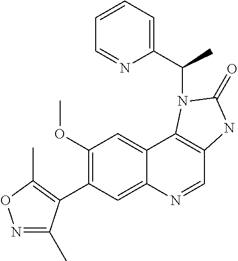 |
| Example 4 | 7-(3,5-dimethyl-4-isoxazolyl)-8- (methoxy)-1-[(1R)-phenylethyl]-2- (tetrahydro-2H-pyran-4-yl)-1H- imidazo[4,5-c]quinoline | 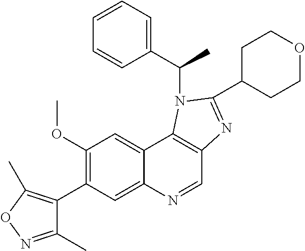 |
| Example 5 | 4-{(2S,4R)-1-acetyl-4-[(4- chlorophenyl)amino]-2-methyl-1,2,3,4- tetrahydro-6-quinolinyl}benzoic acid | 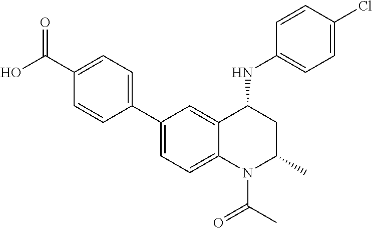 |
| Example 6 | N-{1-methyl-7-[4-(1- piperidinylmethyl)phenyl][1,2,4]triazolo [4,3-a]quinolin-4-yl}urea | 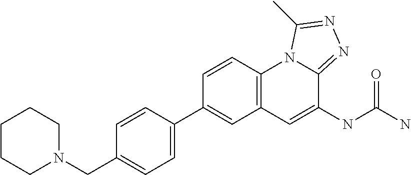 |

Examples 1-6 can be prepared by methods described herein.

In a further embodiment the bromodomain inhibitor is a compound that is generically or specifically disclosed in PCT publication WO2006/032470 (SmithKline Beecham Corporation). Such compounds can be prepared by methods described therein.

In a further embodiment the bromodomain inhibitor is a compound that is generically or specifically disclosed in PCT publication WO2009/084693 (Mitsubishi Tanabe). Such compounds can be prepared by methods described therein.

In a further embodiment the bromodomain inhibitor is 1-[2-(1H-benzimidazol-2-ylthio)ethyl]-1,3-dihydro-3-methyl-2H-benzinidazole-2-thione as described in Japanese patent application JP2008-156311.

It will be appreciated that the bromodomain inhibitor used in the present invention may be in the form of a pharmaceutically acceptable salt, solvate (e.g. a hydrate) or prodrug or any other derivative of such a compound which upon administration to the recipient is capable of providing (directly or indirectly) the bromodomain inhibitor of the invention, or an active metabolite or residue thereof. Suitable pharmaceutically acceptable salts can include acid or base addition salts. For a review on suitable salts see Berge et al., J. Pharm. Sci., 66:1-19, (1977). Typically, a pharmaceutically acceptable salt may be readily prepared by using a desired acid or base as appropriate. The resultant salt may precipitate from solution and be collected by filtration or may be recovered by evaporation of the solvent. Suitable prodrugs are recognizable to those skilled in the art, without undue experimentation. Nevertheless, reference is made to the teaching of Burger's Medicinal Chemistry and Drug Discovery, 5^{th} Edition, Vol 1: Principles and Practice.

In a second aspect of the present invention there is provided a bromodomain inhibitor for use in the treatment of autoimmune and inflammatory diseases or conditions.

In a third aspect of the present invention, there is provided the use of a bromodomain inhibitor in the manufacture of a medicament for the treatment of autoimmune and inflammatory diseases or conditions.

As used herein the term “autoimmune and inflammatory diseases or conditions” is intended to denote a wide variety of chronic autoimmune and inflammatory conditions such as rheumatoid arthritis, osteoarthritis, acute gout, psoriasis, systemic lupus erythematosus, multiple sclerosis, inflammatory bowel disease (Crohn's disease and Ulcerative colitis), asthma, chronic obstructive airways disease, pneumonitis, myocarditis, pericarditis, myositis, eczema, dermatitis, alopecia, vitiligo, bullous skin diseases, nephritis, vasculitis, atherosclerosis, Alzheimer's disease, depression, retinitis, uveitis, scleritis, hepatitis, pancreatitis, primary biliary cirrhosis, sclerosing cholangitis, Addison's disease, hypophysitis, thyroiditis, type I diabetes and acute rejection of transplanted organs.

The term “autoimmune and inflammatory diseases or conditions” is also intended to include acute inflammatory conditions such as acute gout, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's Arteritis, vasculitis with organ involvement and acute rejection of transplanted organs.

The term “autoimmune and inflammatory diseases or conditions” is also intended to include diseases or conditions which involve inflammatory responses to infections with bacteria, viruses, fungi, parasites or their toxins, such as sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria, SIRS associated with viral infections such as influenza, herpes zoster, herpes simplex, coronavirus.

The term “autoimmune and inflammatory diseases or conditions”, is intended to include each of or all of the above disease states.

In one embodiment the disease or condition for which a bromodomain inhibitor is indicated is selected from diseases associated with systemic inflammatory response syndrome, such as sepsis, burns, pancreatitis, major trauma, haemorrhage and ischaemia. In this embodiment the bromodomain inhibitor would be administered at the point of diagnosis to reduce the incidence of: SIRS, the onset of shock, multi-organ dysfunction syndrome, which includes the onset of acute lung injury, ARDS, acute renal, hepatic, cardiac and gastro-intestinal injury and mortality. In another embodiment the bromodomain inhibitor would be administered prior to surgical or other procedures associated with a high risk of sepsis, haemorrhage, extensive tissue damage, SIRS or MODS (multiple organ dysfunction syndrome). In a particular embodiment the bromodomain inhibitor is indicated for the treatment of sepsis, sepsis syndrome, septic shock or endotoxaemia. In another embodiment, the bromodomain inhibitor is indicated for the treatment of acute or chronic pancreatitis. In another embodiment the bromodomain is indicated for the treatment of burns.

While it is possible that for use in therapy, the bromodomain inhibitor may be administered as the raw chemical, it is common to present the active ingredient as a pharmaceutical composition.

In a fourth aspect the present invention provides a pharmaceutical formulation comprising a bromodomain inhibitor and at least one pharmaceutical carrier, diluent or excipient, wherein the bromodomain inhibitor is present in an amount effective for use in the treatment of autoimmune and inflammatory diseases or conditions.

The carrier(s), diluent(s) or excipient(s) must be acceptable in the sense of being compatible with the other ingredients of the composition and not deleterious to the recipient thereof.

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

Pharmaceutical compositions may be adapted for administration by any appropriate route, for example by the oral (including buccal or sublingual), rectal, inhaled, intranasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

In one embodiment the pharmaceutical composition is adapted for parenteral administration, particularly intravenous administration.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders suitable for incorporating into tablets or capsules may be prepared by reducing the compound to a suitable fine size (e.g. by micronisation) and mixing with a similarly prepared pharmaceutical carrier such as an edible carbohydrate, as, for example, starch or mannitol. Flavoring, preservative, dispersing and coloring agent can also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release as for example by coating or embedding particulate material in polymers, wax or the like.

The bromodomain inhibitor can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Dosage forms for nasal or inhaled administration may conveniently be formulated as aerosols, solutions, suspensions, gels or dry powders.

For compositions suitable and/or adapted for inhaled administration, it is preferred that the compound of the invention is in a particle-size-reduced form e.g. obtained by micronisation. The preferable particle size of the size-reduced (e.g. micronised) compound or salt is defined by a D50 value of about 0.5 to about 10 microns (for example as measured using laser diffraction).

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol.

For pharmaceutical compositions suitable and/or adapted for inhaled administration, the pharmaceutical composition may be a dry powder inhalable composition. Such a composition can comprise a powder base such as lactose, glucose, trehalose, mannitol or starch, the compound of formula (I) or salt thereof (preferably in particle-size-reduced form, e.g. in micronised form), and optionally a performance modifier such as L-leucine or another amino acid and/or metals salts of stearic acid such as magnesium or calcium stearate. Preferably, the dry powder inhalable composition comprises a dry powder blend of lactose e.g. lactose monohydrate and the compound of formula (I) or salt thereof. Such compositions can be administered to the patient using a suitable device such as the DISKUS® device, marketed by GlaxoSmithKline which is for example described in GB 2242134A.

The bromodomain inhibitor may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO-A-2005/044354.

A therapeutically effective amount of a compound of the bromodomain inhibitor will depend upon a number of factors including, for example, the age and weight of the animal, the precise condition requiring treatment and its severity, the nature of the formulation, and the route of administration, and will ultimately be at the discretion of the attendant physician or veterinarian. In the pharmaceutical composition, each dosage unit for oral or parenteral administration preferably contains from 0.01 to 3000 mg, more preferably 0.5 to 1000 mg, of a compound of the invention calculated as the free base. Each dosage unit for nasal or inhaled administration preferably contains from 0.001 to 50 mg, more preferably 0.01 to 5 mg, of a compound of the formula (I) or a pharmaceutically acceptable salt thereof, calculated as the free base.

The compounds for use in the invention can be administered in a daily dose (for an adult patient) of, for example, an oral or parenteral dose of 0.01 mg to 3000 mg per day or 0.5 to 1000 mg per day, or a nasal or inhaled dose of 0.001 to 50 mg per day or 0.01 to 5 mg per day, of the compound of the formula (I) or a pharmaceutically acceptable salt thereof, calculated as the free base. This amount may be given in a single dose per day or more usually in a number (such as two, three, four, five or six) of sub-doses per day such that the total daily dose is the same. An effective amount of a salt thereof, may be determined as a proportion of the effective amount of the compound of formula (I) per se.

The compounds for use in the invention may be employed alone or in combination with other therapeutic agents. Combination therapies according to the present invention thus comprise the administration of bromodomain inhibitor and the use of at least one other pharmaceutically active agent. Preferably, combination therapies according to the present invention comprise the administration of at least one bromodomain inhibitor and at least one other pharmaceutically active agent. The bromodomain inhibitor and the other pharmaceutically active agent(s) may be administered together in a single pharmaceutical composition or separately and, when administered separately this may occur simultaneously or sequentially in any order. The amounts of the bromodomain inhibitor and the other pharmaceutically active agent(s) and the relative timings of administration will be selected in order to achieve the desired combined therapeutic effect. Thus in a further aspect, there is provided a combination comprising a bromodomain inhibitor and at least one other pharmaceutically active agent.

Thus in one aspect, the pharmaceutical compositions according to the invention may be used in combination with or include one or more other therapeutic agents, for example selected from antibiotics, anti-virals, glucocorticosteroids, muscarinic antagonists, beta-2 agonists, NSAIDs and anti-TNFα agents.

It will be appreciated that when the bromodomain inhibitor is administered in combination with other therapeutic agents normally administered by the inhaled, intravenous, oral or intranasal route, that the resultant pharmaceutical composition may be administered by the same routes. Alternatively the individual components of the composition may be administered by different routes.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The identification of novel bromodomain inhibitors may lead to more effective drugs for the treatment of autoimmune and inflammatory diseases or conditions. Accordingly in a yet further aspect the invention provides a method for identifying compounds for use in treating autoimmune and inflammatory diseases or conditions which comprises the step of determining whether the compound inhibits the binding of a bromodomain with its cognate acetylated protein. In one embodiment there is provided a method for identifying compounds for use in treating autoimmune and inflammatory diseases or conditions which comprises the step of determining whether the compound is a BET family bromodomain inhibitor.

Suitable screening methods are familiar to those skilled in the art and include fluorescence (e.g. FRET) and radioligand binding techniques.

There is further provided a method for identifying compounds for use in treating autoimmune and inflammatory diseases or conditions which comprises a competitive binding assay between the compound to be determined whether it inhibits the binding of a bromodomain with its cognate acetylated protein and a fluorescent or radioligand derivative of a bromodomain inhibitor. In one embodiment the fluorescent or radioligand derivative of a bromodomain inhibitor is a fluorescent derivative of a bromodomain inhibitor described herein, such as Examples 1-6. In a yet further embodiment the fluorescent derivative of a bromodomain inhibitor is Reference compound C. Reference compound C is believed to be novel.

The present invention also provides a compound for use in treating autoimmune and inflammatory diseases or conditions identified by the method described above.

Certain compounds for use in this invention invention may be prepared by the methods described below or by similar methods.

### General Experimental Details

All temperatures referred to are in ° C.

### Abbreviations

AcCl refers to acetyl chloride
AcOH refers to acetic acid
BINAP refers to 2,2′-bis(diphenylphosphino)-1,1′-binaphthyl
CV refers to column volumes
DCM refers to dichloromethane
1,2-DCE refers to 1,2-dichloroethane
DME refers to dimethoxyethane
DMF refers to N,N-dimethylformamide
DMSO refers to dimethylsulfoxide.
Ether refers to diethyl ether
Et₂O refers to diethyl ether
Et₃N refers to triethylamine
EtOAc refers to ethyl acetate
FMOC refers to 9-fluorenylmethoxycarbonyl
HPLC refers to high performance liquid chromatography
HRMS refers to high resolution mass spectroscopy
i-Pr₂O refers to di-isopropyl ether
MDAP or Mass directed autoprep refers preparative mass directed HPLC
MeCN refers to acetonitrile
MeOH refers to methanol
Mp refers to melting point
Rf refers to retention factor
r.t. refers to room temperature
Rt refers to retention time
Na₂SO₄ refers to sodium sulfate
TFA refers to trifluoroacetic acid
THF refers to tetrahydrofuran
TLC refers to thin layer chromatography

Experimental details of LC-MS methods A-F as referred to herein are as follows:

LC/MS (Method A) was conducted on an Acquity HPLC BEH C18 column (50 mm×2.1 mm i.d. 1.7 μm packing diameter) at 40 degrees centigrade, eluting with 10 mM Ammonium Bicarbonate in water adjusted to pH 10 with Ammonia solution (Solvent A) and Acetonitrile (Solvent B) using the following elution gradient 0-1.5 min 1-97% B, 1.5-1.9 min 97% B, 1.9-2.0 min 100% B at a flow rate of 1 ml/min. The UV detection was a summed signal from wavelength of 210 nm to 350 nm. The mass spectra were recorded on a Waters ZQ Mass Spectrometer using Alternate-scan Positive and Negative Electrospray. Ionisation data was rounded to the nearest integer.

LC/MS (Method B) was conducted on an Acquity HPLC BEH C18 column (50 mm×2.1 mm i.d. 1.7 μm packing diameter) at 40 degrees centigrade, eluting with 0.1% v/v solution of formic acid in water (Solvent A) and 0.1% v/v solution of formic acid in acetonitrile (Solvent B) using the following elution gradient 0-1.5 min 3-100% B, 1.5-1.9 min 100% B, 1.9-2.0 min 3% B at a flow rate of 1 ml/min. The UV detection was a summed signal from wavelength of 210 nm to 350 nm. The mass spectra were recorded on a Waters ZQ Mass Spectrometer using Alternate-scan Positive and Negative Electrospray. Ionisation data was rounded to the nearest integer.

LC/MS (Method C) was conducted on an Acquity HPLC BEH C18 column (50 mm×2.1 mm i.d. 1.7 μm packing diameter) at 40 degrees centigrade, eluting with 0.1% v/v solution of trifluoroacetic acid in water (Solvent A) and 0.1% v/v solution of trifluoroacetic acid in acetonitrile (Solvent B) using the following elution gradient 0-1.5 min 3-100% B, 1.5-1.9 min 100% B, 1.9-2.0 min 3% B at a flow rate of 1 ml/min. The UV detection was a summed signal from wavelength of 210 nm to 350 nm. The mass spectra were recorded on a Waters ZQ Mass Spectrometer using Positive Electrospray. Ionisation data was rounded to the nearest integer.

LC/MS (Method D) was conducted on a Supelcosil LCABZ+PLUS column (3 μm, 3.3 cm×4.6 mm ID) eluting with 0.1% HCO₂H and 0.01 M ammonium acetate in water (solvent A), and 95% acetonitrile and 0.05% HCO₂H in water (solvent B), using the following elution gradient 0-0.7 minutes 0% B, 0.7-4.2 minutes 0→100% B, 4.2-5.3 minutes 100% B, 5.3-5.5 minutes 100→0% B at a flow rate of 3 mL/minute. The mass spectra (MS) were recorded on a Fisons VG Platform mass spectrometer using electrospray positive ionisation [(ES+ve to give [M+H]⁺ and [M+NH₄]⁺ molecular ions] or electrospray negative ionisation [(ES-ve to give [M−H]− molecular ion] modes. Analytical data from this apparatus are given with the following format: [M+H]⁺ or [M−H]⁻.

LC/MS (Method E) was conducted on a Chromolith Performance RP 18 column (100×4.6 mm id) eluting with 0.01 M ammonium acetate in water (solvent A) and 100% acetonitrile (solvent B), using the following elution gradient 0-4 minutes 0-100% B, 4-5 minutes 100% B at a flow rate of 5 ml/minute. The mass spectra (MS) were recorded on a micromass Platform-LC mass spectrometer using atmospheric pressure chemical positive ionisation [AP+ve to give MH+ molecular ions] or atmospheric pressure chemical negative ionisation [AP−ve to give (M−H)− molecular ions] modes. Analytical data from this apparatus are given with the following format: [M+H]+ or [M−H]−.

LC/MS (Method F) was conducted on an Sunfire C18 column (30 mm×4.6 mm i.d. 3.5 μm packing diameter) at 30 degrees centigrade, eluting with 0.1% v/v solution of Trifluoroacetic Acid in Water (Solvent A) and 0.1% v/v solution of Trifluoroacetic Acid in Acetonitrile (Solvent B) using the following elution gradient 0-0.1 min 3% B, 0.1-4.2 min 3-100% B, 4.2-4.8 min 100% B, 4.8-4.9 min 100-3% B, 4.9-5.0 min 3% B at a flow rate of 3 ml/min. The UV detection was an averaged signal from wavelength of 210 nm to 350 nm and mass spectra were recorded on a mass spectrometer using positive electrospray ionization. Ionisation data was rounded to the nearest integer.

LC/HRMS: Analytical HPLC was conducted on a Uptisphere-hsc column (3 μm 33×3 mm id) eluting with 0.01M ammonium acetate in water (solvent A) and 100% acetonitrile (solvent B), using the following elution gradient 0-0.5 minutes 5% B, 0.5-3.75 minutes 5→100% B, 3.75-4.5 100% B, 4.5-5 100→5% B, 5-5.5 5% B at a flow rate of 1.3 mL/minute. The mass spectra (MS) were recorded on a micromass LCT mass spectrometer using electrospray positive ionisation [ES+ve to give MH⁺ molecular ions] or electrospray negative ionisation [ES−ve to give (M−H)⁻ molecular ions] modes.

TLC (thin layer chromatography) refers to the use of TLC plates sold by Merck coated with silica gel 60 F254.

“Mass directed autoprep”/“preparative mass directed HPLC” was conducted on a system such as; a Waters FractionLynx system comprising of a Waters 600 Gradient pump, a Waters 2767 inject/collector, a Waters Reagent manager, a Gilson Aspec—waste collector, a Gilson 115 post-fraction UV detector and a Computer System. The column used is typically a Supelco LCABZ++ column whose dimensions are 20 mm internal diameter by 100 mm in length. The stationary phase particle size is 5 μm. A flow rate was used of 20 mL/min with either 0.1% formic acid or trifluoroacetic acid in water (solvent A) and 0.1% formic or trifluoroacetic acid in acetonitrile (solvent B) using the appropriate elution gradient. Mass spectra were recorded on Micromass ZQ mass spectrometer using electrospray positive and negative mode, alternate scans. The software used was MassLynx 4.0 or using equivalent alternative systems.

Silica chromatography techniques include either automated (Flashmaster or Biotage SP4) techniques or manual chromatography on pre-packed cartridges (SPE) or manually-packed flash columns.

Microwave chemistry was typically performed in sealed vessels, irradiating with a suitable microwave reactor system, such as a Biotage Initiator™ Microwave Synthesiser.

When the name of a commercial supplier is given after the name of a compound or a reagent, for instance “compound X (Aldrich)” or “compound X/Aldrich”, this means that compound X is obtainable from a commercial supplier, such as the commercial supplier named.

Similarly, when a literature or a patent reference is given after the name of a compound, for instance compound Y (EP 0 123 456), this means that the preparation of the compound is described in the named reference.

The names of the above mentioned Examples have been obtained using the compound naming programme “ACD Name Pro 6.02”.

### Intermediate 1

### [(4S)-6-(4-Chlorophenyl)-1-methyl-8-(methyloxy)-4H-[1,2,4]triazolo[4,3-a][1,4]benzodiazepin-4-yl]acetic acid

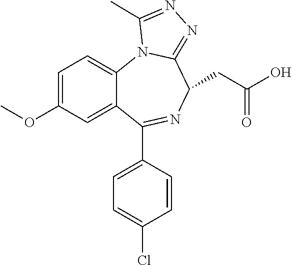

To a solution of Intermediate 2 (7.4 g, 18.1 mmol) in THF (130 mL) at r.t. was added 1N NaOH (36.2 mL, 36.2 mmol). The reaction mixture was stirred at this temperature for 5 h before being quenched with 1N HCl (36.2 mL) and concentrated in vacuo. Water is then added and the aqueous layer was extracted with DCM (×3) and the combined organic layers were dried over Na₂SO₄, filtered and concentrated under reduced pressure to give the title compound (7 g, 98% yield) as a pale yellow solid.

LC/MS (Method D): m/z 397 [M+H]⁺

### Intermediate 2

### Methyl[(4S)-6-(4-chlorophenyl)-1-methyl-8-(methyloxy)-4H-[1,2,4]triazolo[4,3-a][1,4]benzodiazepin-4-yl]acetate

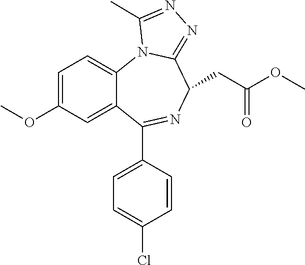

The crude Intermediate 3 (assumed 9.7 g) was suspended in THF (100 ml) and AcOH (60 mL) was added at r.t. The reaction mixture was stirred at this temperature for 2 days before being concentrated under reduced pressure. The crude solid was triturated in i-Pr₂O and filtered to give the title compound (8.7 g, 91% over 3 steps) as an off-white solid. HRMS (M+H)⁺ calculated for C₂₁H₂₀ClN₄O₃ 411.1229. found 411.1245.

### Intermediate 3

### Methyl[(3S)-2-[(1Z)-2-acetylhydrazino]-5-(4-chlorophenyl)-7-(methyloxy)-3H-1,4-benzodiazepin-3-yl]acetate

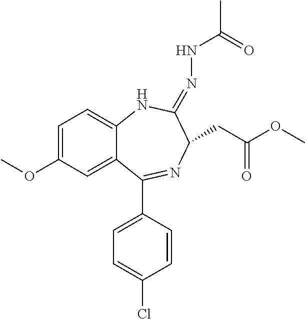

To a suspension of Intermediate 4 (9.0 g, 23.2 mmol) in THF (300 mL) at 0° C. was added hydrazine monohydrate (3.4 mL, 69.6 mmol) dropwise. The reaction mixture was stirred for 5 h between 5° C. and 15° C. before being cooled at 0° C. Et₃N (9.7 mL, 69.6 mmol) was then added slowly and AcCl (7.95 mL, 69.6 mmol) was added dropwise. The mixture was then allowed to warm to r.t. for 16 h before being concentrated under reduced pressure. The crude product was dissolved in DCM and washed with water. The organic layer was dried over Na₂SO₄, filtered and concentrated in vacuo to give the crude title compound (9.7 g, 98% yield) which was used without further purification.

R_{f}=0.49 (DCM/MeOH:90/10).

### Intermediate 4

### Methyl[(3S)-5-(4-chlorophenyl)-7-(methyloxy)-2-thioxo-2,3-dihydro-1H-1,4-benzodiazepin-3-yl]acetate

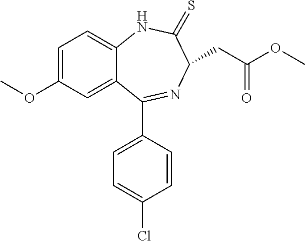

A suspension of P₄S₁₀ (36.1 g, 81.1 mmol) and Na₂CO₃ (8.6 g, 81.1 mmol) in 1,2-DCE (700 mL) at r.t. was stirred for 2 h before Intermediate 5 (16.8 g, 45.1 mmol) was added.

The resulting mixture was stirred at 70° C. for 2 h before being cooled and filtered. The solid was washed twice with DCM and the filtrate washed with sat. NaHCO₃ and brine. The organic layer was dried over Na₂SO₄, filtered and concentrated under reduced pressure. The crude product was purified by flash-chromatography on silica gel (DCM/MeOH:99/1) to afford the title compound (17.2 g, 98% yield) as a yellowish solid.

LC/MS (Method D): m/z 389 [M(³⁵Cl)+H]⁺, Rt 2.64 min.

HRMS (M+H)⁺ calculated for C₁₉H₁₈³⁵ClN₂O₃S 389.0727. found 389.0714.

### Intermediate 5

### Methyl[(3S)-5-(4-chlorophenyl)-7-(methyloxy)-2-oxo-2,3-dihydro-1H-1,4-benzodiazepin-3-yl]acetate

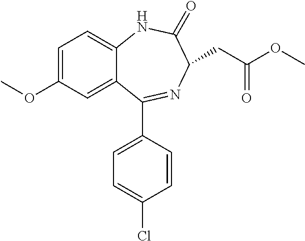

To a solution of the crude Intermediate C12 (assumed 0.2 mol) in DCM (500 mL) was added Et₃N (500 mL, 3.65 mol) and the resulting mixture was refluxed for 24 h before being concentrated. The resulting crude amine was dissolved in 1,2-DCE (1.5 L) and AcOH (104 mL, 1.8 mol) was added carefully. The reaction mixture was then stirred at 60° C. for 2 h before being concentrated in vacuo and dissolved in DCM. The organic layer was washed with 1N HCl and the aqueous layer was extracted with DCM (×3). The combined organic layers were washed twice with water, and brine, dried over Na₂SO₄, filtered and concentrated under reduced pressure. The crude solid was recrystallised in MeCN leading to the title compound (51 g) as a pale yellow solid.

R_{f}=0.34 (DCM/MeOH:95/5). HRMS (M+H)⁺ calculated for C₁₉H₁₈³⁵ClN₂O₄ 373.0955. found 373.0957.

### Intermediate 6

### Methyl N¹-[2-[(4-chlorophenyl)carbonyl]-4-(methyloxy)phenyl]-N²-{[(9H-fluoren-9-ylmethyl)oxy]carbonyl}-L-α-asparaginate

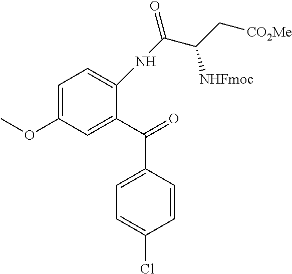

Methyl N-{[(9H-fluoren-9-ylmethyl)oxy]carbonyl}-L-α-aspartyl chloride (Int. J. Peptide Protein Res. 1992, 40, 13-18) (93 g, 0.24 mol) was dissolved in CHCl₃ (270 mL) and Intermediate 7 (53 g, 0.2 mol) was added. The resulting mixture was stirred at 60° C. for 1 h before being cooled and concentrated at 60% in volume. Ether was added at 0° C. and the resulting precipitate was filtered and discarded. The filtrate was concentrated under reduced pressure and used without further purification.

### Intermediate 7

### [2-amino-5-(methyloxy)phenyl](4-chlorophenyl)methanone

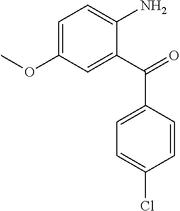

To a solution of Intermediate 8 (40.0 g, 0.21 mol) in a toluene/ether (2/1) mixture (760 mL) at 0° C. was added dropwise a solution of 4-chlorophenylmagnesium bromide (170 mL, 1M in Et₂O, 0.17 mol). The reaction mixture was allowed to warm to r.t. and stirred for 1 h before being quenched with 1N HCl (200 mL). The aqueous layer was extracted with EtOAc (3×150 mL) and the combined organics were washed with brine (100 mL), dried over Na₂SO₄, filtered and concentrated under reduced pressure. The crude compound was then dissolved in EtOH (400 mL) and 6N HCl (160 mL) was added. The reaction mixture was refluxed for 2 h before being concentrated to one-third in volume. The resulting solid was filtered and washed twice with ether before being suspended in EtOAc and neutralised with 1N NaOH. The aqueous layer was extracted with EtOAc (3×150 mL) and the combined organics were washed with brine (150 mL), dried over Na₂SO₄, filtered and concentrated under reduced pressure. The title compound was obtained as a yellow solid (39 g, 88% yield). LC/MS (Method D): m/z 262 [M+H]+, Rt 2.57 min.

### Intermediate 8

### 2-methyl-6-(methyloxy)-4H-3,1-benzoxazin-4-one

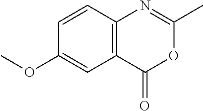

A solution of 5-methoxyanthranilic acid (Lancaster) (41.8 g, 0.25 mol) was refluxed in acetic anhydride (230 mL) for 3.5 h before being concentrated under reduced pressure. The crude compound was then concentrated twice in the presence of toluene before being filtered and washed twice with ether to yield to the title compound (33.7 g, 71% yield) as a brown solid. LC/MS (Method D): m/z 192 [M+H]⁺, Rt 1.69 min

### Intermediate 9

### 4-chloro-7-(3,5-dimethyl-4-isoxazolyl)-6-(methoxy)-3-quinolinecarboxamide

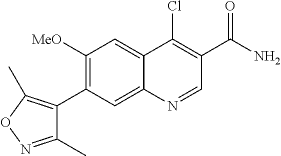

A mixture of Intermediate 10 (27.7 g, 0.079 mol) and 10 drops of anhydrous DMF in POCl₃ (250 mL) was refluxed for 5 hours. Then, the mixture was concentrated to dryness under vacuum. The residue was treated twice with 100 mL of toluene and evaporated to dryness to remove the last traces of POCl₃. The dry foam obtained is added portionwise to an aqueous solution of ammonia (25%, 300 ml) cooled to 0/5° C. with an ice bath. After the end of the addition, the vigourous stirring was maintained for 1 hour at this temperature. Then, the brown solid material was filtered off and washed respectively with water (3×200 mL), diisopropyl ether (2×200 mL) and pentane (100 mL) to give after drying the crude product. This material was purified by a flash chromatography on silica gel (eluant=CH₂Cl₂/MeOH, 95/5) to give the title compound (16.8 g, 64.7%).

¹H NMR (300 MHz, CDCl3, ppm) δ: 8.96 (s, 1H), 7.86 (s, 1H), 7.54 (s, 1H), 3.95 (s, 3H), 2.30 (s, 3H), 2.15 (s, 3H).

### Intermediate 10

### 7-(3,5-dimethyl-4-isoxazolyl)-4-hydroxy-6-(methyloxy)-3-quinolinecarboxylic acid

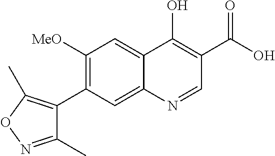

A mixture of Intermediate 11 (5.6 g, 16.4 mmol), ethanol (40 ml) and sodium hydroxide (1M solution, 16.4 ml) was combined in a round bottom flask and heated to 100° C. for 16 h. Additional sodium hydroxide (10 ml conc. NaOH solution) and 10 ml water were added and reaction mixture continued to reflux at 100° C. Reaction mixture was evaporated to dryness and taken up in water. 1M HCl was added dropwise until a precipitate formed. This was filtered and dried in a vacuum dessicator to give the title compound as a beige solid (4.6 g, 80%). LC/MS (Method E): m/z 315 [M+H]⁺

### Intermediate 11

### Ethyl 7-(3,5-dimethyl-4-isoxazolyl)-4-hydroxy-6-(methyloxy)-3-quinolinecarboxylate

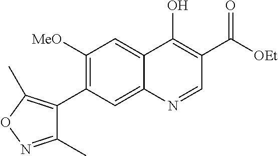

In a 3 neck 1 L r.b.f. was added diphenyl ether (350 ml) and heated to 260 deg. C. Intermediate 12 was added (40 g) in a single portion and reaction was heated with Dean Stark apparatus under Nitrogen for 30 mins. The reaction mixture was cooled down to 170° C., the solvent was distilled using a pump. The reaction mixture was then cooled down to 80° C., poured into a solution of 1:1 diisopropyl ether/cyclohexane (500 mL). The solid was filtered, washed with diisopropyl ether and pentane and dried at 50° C. to give the title compound as a brown solid (28 g, 79%). LC/MS (Method E): m/z 343.11 [M+H]⁺

### Intermediate 12

### Diethyl({[3-(3,5-dimethyl-4-isoxazolyl)-4-(methyloxy)phenyl]amino}methyl idene)propanedioate

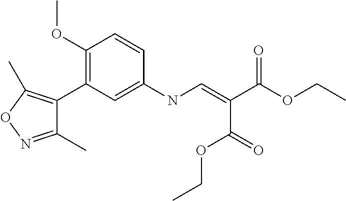

A mixture of Intermediate 13 (107.8 g, 0.50 mol), diethyl ethoxymethylenemalonate (105 ml, 0.518 mol) was heated at 130° C. with Dean-Stark apparatus. After 45 mins, ethanol (15 ml) was removed. The temperature was adjusted to 75° C. and poured into diisopropyl ether (500 mL). Reaction mixture was stirred and solid precipitate was filtered and washed with diisopropyl ether to give the title compound as a brown solid (169.5 g, 88%).

¹H NMR (400 MHz, Chloroform-d, ppm) δ: 11.0 (1H, s), 8.4 (1H, d), 7.2 (1H, dd), 7.0 (1H, d), 6.9 (1H, d), 4.3 (2H, q), 4.25 (2H, q), 3.8 (3H, s), 2.3 (3H, s), 2.2 (3H, s), 1.4 (3H, t), 1.35 (3H, t)

### Intermediate 13

### 3-(3,5-Dimethyl-4-isoxazolyl)-4-(methoxy)aniline

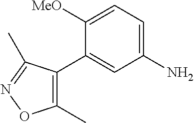

To a solution of Intermediate 14 (1.7 g, 6.85 mmol, 1 eq.) in EtOH (170 mL), was added Pd/C (10% on carbon, 85 mg) and the reaction was stirred under hydrogen for 4 hours. AcOH (1.7 mL) was added and the reaction was hydrogenated for 20 hours. After filtration, the solvent was evaporated in vacuo. The crude compound was dissolved into DCM and washed with saturated aqueous NaHCO₃, dried over Na₂SO₄, filtered and evaporated. The title compound was obtained as a red oil (1.38 g, 88%). GC/MS m/z: 218.

### Intermediate 14

### 3,5-Dimethyl-4-[2-(methoxy)-5-nitrophenyl]isoxazole

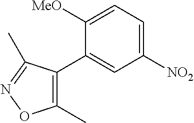

To a solution of 2-iodo-1-(methoxy)-4-nitrobenzene (2 g, 7.17 mmol, 1 eq.) and (3,5-dimethylisoxazole)boronic acid (3.03 g, 21.5 mmol, 3 eq.) in DME (44 mL) and water (7 mL) were added tetrakis(triphenylphosphine) palladium(0) (0.415 g, 0.05 eq.) and Ba(OH)₂.8H₂O (4.52 g, 14.33 mmol, 2 eq.). The mixture was heated at 80° C. for 16 hours. To complete the reaction (3,5-dimethylisoxazole)boronic acid (1 eq.) was added and the mixture was heated for 4 hours. The cooled mixture was filtered and extracted with DCM. The organic phase was washed with saturated aqueous NaHCO₃ and water, dried over Na₂SO₄ and filtered. Evaporation of the solvent in vacuo gave a crude oil which was precipitated with i-Pr₂O to afford the title compound as a rust coloured solid (1.735 g, 97%). GC/MS m/z: 248

### Intermediate 15

### 7-(3,5-dimethyl-4-isoxazolyl)-6-(methoxy)-N⁴-[(1R)-1-phenylethyl]-3,4-quinolinediamine

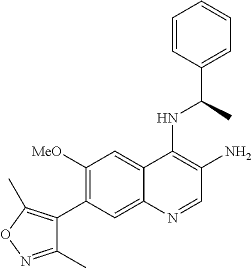

A mixture of Intermediate 16 (2.5 g, 7.5 mmol) and (R)-(+)-alpha-methylbenzylamine (2 eq, 1.82 g, Aldrich) in CH₃CN (30 mL) was heated at 60° C. for 2 hours. The mixture was extracted with DCM. The organic phase washed with saturated aqueous NaHCO₃ and dried over Na₂SO₄. The solvent was evaporated under reduce pressure and the residue taken up in diethyl ether. The precipitate was filtered off and died under vacuo to give 7-(3,5-dimethyl-4-isoxazolyl)-6-(methoxy)-3-nitro-N-[(1R)-1-phenylethyl]-4-quinolinamine (2.5 g) which was used without purification in the next step.

To a solution of this nitro intermediate (2.5 g, 24.82 mmol) in a mixture of EtOH (20 mL) and HCl (3.8 mL), was added portionwise SnCl₂,2H₂O (5.6 g, 24.82 mmol). The reaction mixture was heated to 40° C. for 1 hour, then hydrolysed with NaOH N and extracted with DCM. The organic phase was washed with water, dried and concentrated to give the title compound as a brown powder (0.5 g, 17%).

LCMS (Method E): m/z: 389 (M+H)⁺, Rt=2.95 min.

### Intermediate 16

### 4-chloro-7-(3,5-dimethyl-4-isoxazolyl)-6-(methoxy)-3-nitroquinoline

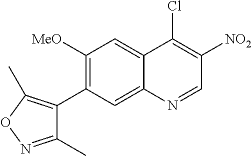

A suspension of Intermediate 17 (5 g, 16 mmol) in POCl₃ (20 mL) was refluxed overnight. After cooling, the mixture was evaporated to dryness. The resulting residue was poured over saturated aqueous NaHCO₃ and extracted with DCM. The organic layer was washed with water, dried over Na₂SO₄. The solvent was evaporated under reduced pressure to give the title compound as a light brown powder (5 g, 94%).

¹H NMR (300 MHz, DMSO-d6, ppm) δ: 9.27 (s, 1H), 8.15 (s, 1H), 7.73 (s, 1H), 4.05 (s, 3H), 2.36 (s, 3H), 2.16 (s, 3H).

### Intermediate 17

### 7-(3,5-dimethyl-4-isoxazolyl)-6-(methoxy)-3-nitro-4-quinolinol

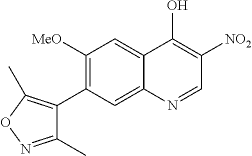

Nitric acid (10 mL) was added slowly to a solution of Intermediate 18 (28 g, 104 mmol) in propanoic acid (450 mL) at room temperature, followed by heating the reaction mixture to 100° C. for 1 h. After cooling with an ice bath, the precipitate was filtered off, washed with pentane to give the title compound as a yellow powder (27 g, 82%).

LCMS (Method E): m/z: 314 (MH⁻), Rt=2.12 min. ¹H NMR (300 MHz, DMSO-d6, ppm) δ: 13.06 (s, 1H), 9.26 (s, 1H), 7.84 (s, 1H), 7.67 (s, 1H), 3.98 (s, 3H), 2.39 (s, 3H), 2.19 (s, 3H).

### Intermediate 18

### 7-(3,5-dimethyl-4-isoxazolyl)-4-quinolinol

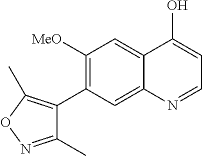

3,5-dimethylisoxazole-4-boronic acid (49.2 g, 0.349 mol) and Ba(OH)₂.8H₂O (91.8 g, 0.291 mol, Acros) were added to a solution of Intermediate 19 (35 g, 0.116 mol) in a mixture of water (180 mL)) and 1,2-dimethoxyethane (600 mL). The reaction was put under nitrogen for 15 minutes and tetrakis(triphenylphosphine)palladium(0) was added (4.1 g, 3.55 mmol, Aldrich). The reaction mixture was stirred at 105° C. overnight. After cooling at room temperature, the mixture was poured into water and extracted with ethyl acetate. The aqueous layer was acidified to pH 7 with concentrated HCl and extracted with ethyl acetate. The aqueous layer was basified to pH 10 with NaOH 5N and extracted with ethyl acetate. The organic layers were combined, washed with a saturated aqueous NaCl and dried. The crude brown oil was then purified by flash chromatography on silicagel eluting with DCM/MeOH (9:1) to give the title compound as a brown solid (31.4 g, 43.9%)

¹H NMR (300 MHz, DMSO-d6, ppm) δ: 11.72 (bs, 1H), 7.88 (d, J=7.7 Hz, 1H), 7.61 (s, 1H), 7.42 (s, 1H), 6.03 (d, J=7.3 Hz, 1H), 3.86 (s, 3H), 2.31 (s, 1H), 2.11 (s, 1H).

### Intermediate 19

### 6-iodo-7-(methoxy)-1-quinolinol

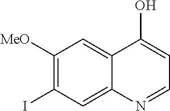

Intermediate 20 (200 g, 0.496 mol) was added to diphenylether (2 L) at 260° C. The reaction mixture was stirred at 260° C. for 10 minutes. The black solution was then cooled at 100° C. and poured into diisopropyl ether (8 L) previously cooled at 0° C. The precipitate was filtered off, poured into cyclohexane (1 L) and then heated to reflux for 1 hour. The solid was filtered off, poured into methanol (250 mL) and heated at 45° C. for 15 minutes. The solid was then filtered off and dried over pallets pump to give the title compound (105 g, 70%).

¹H NMR (300 MHz, DMSO-d6, ppm) δ ¹H NMR (300 MHz, d₆-DMSO, ppm) δ: 8.07 (s, 1H), 7.88 (d, J=7.3 Hz, 1H), 7.45 (s, 1H), 6.06 (d, J=7.3 Hz, 1H), 3.90 (s, 3H).

### Intermediate 20

### 5-({[3-iodo-4-(methoxy)phenyl]amino}methylidene)-2,2-dimethyl-1,3-dioxane-4,6-dione

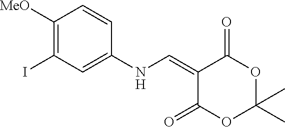

A mixture of 2,2-dimethyl-1,3-dioxane-4,6-dione (203 g, 1.4 mol) and trimethoxymethane (1.5 L) was heated to reflux for 1 hour, then 3-iodo-4-methoxy-aniline (349.2 g, 1.402 mol) was added portionwise. The reaction mixture was stirred at reflux for 1 hour, then cooled to room temperature. The resulting precipitate was filtered off, washed with diisopropyl ether and dried to give the title compound as a beige powder (485 g, 85.9%)

¹H NMR (300 MHz, DMSO-d₆, ppm) δ: 11.2 (d, J=14.6 Hz, 1H), 8.50-8.39 (m, 1H), 8.05 (d, J=2.7 Hz, 1H), 7.60 (dd, J=8.9, 2.7 Hz, 1H), 7.05 (d, J=8.9 Hz, 1H), 3.84 (s, 3H), 1.67 (s, 6H).

### Intermediate 21

### Methyl 4-{(2S,4R)-1-acetyl-4-[(4-chlorophenyl)amino]-2-methyl-1,2,3,4-tetrahydro-6-quinolinyl}benzoate

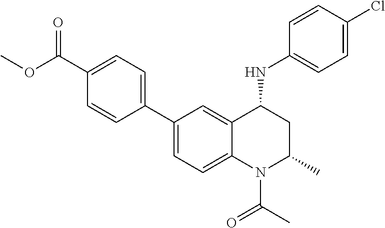

To a flask charged with the Intermediate 22 (800 mg, 2.4 mmol) in toluene (20 mL) was added 4-chlorobromobenzene (501 mg, 2.6 mmol), Pd₂(dba)₃ (87 mg, 0.09 mmol), NaO^{t}Bu (319 mg, 3.3 mmol) and 2′-(dicyclohexylphosphanyl)-N,N-dimethyl-2-biphenylamine (74 mg, 0.19 mmol). The resulting mixture was stirred to 80° C. during 16 hours and 3 additional hours at reflux. The mixture was poured into water and was made acidic upon addition of 1N HCl. Extraction was carried out with EtOAc (2×75 ml) and the organic layers were washed with water and dried over Na₂SO4. After filtration, concentration under reduced pressure and purification by column chromatography eluting with C₆H₁₂/EtOAc:80/20 the title compound was obtained as a white solid (350 mg).

1H NMR (300 MHz, CHLOROFORM-d) δ ppm 1.2 (d, 3H) 1.35 (m, 1H) 2.25 (s, 3H) 2.7 (m, 1H) 3.95 (s, 3H), 4.25 (m, 1H) 4.95 (m, 1H) 6.6 (d, 2H) 7.15 (d, 2H) 7.25 (s, 1H) 7.55 (m, 4H), 8.1 (d, 2H)

LC/MS (Method D): m/z 449 [M+H]⁺ and 447 [M−H]⁻ Rt=3.67 min. [α]_{D}=+326 (c=0.98 g/cl, EtOH)

The title compound eluted at 22.58 min by HPLC as the second peak using a CHIRACEL OD (250×4.6 mm 10 μm) column with hexane/ethanol 90/10 as the mobile phase. A 1 ml/mn flow rate was applied and 10 μL of sample prepared with the dilution of 1 mg of the title compound in 1 ml of eluent was injected. Detection of the compound was carried out with both 210 and 254 nM UV wavelengths. The other enantiomer came off at 15.46 min.

### Intermediate 22

### Methyl 4-[(2S,4R)-1-acetyl-4-amino-2-methyl-1,2,3,4-tetrahydro-6-quinolinyl]benzoate

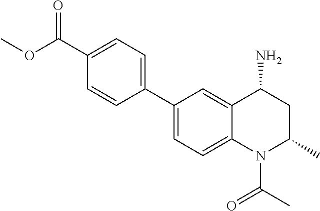

A mixture of Intermediate 23 (121 g) in DCM (3 L) was made basic with addition of an aqueous solution of Na₂CO₃. The resulting free amine was extracted with DCM (2 L) washed with water and dried over Na₂SO₄ to deliver the title compound as an off white solid (79 g).

1H NMR (300 MHz, CHLOROFORM-d) δ ppm 1.15 (m, 4H) 1.7 (m, 2H) 2.15 (s, 3H) 2.6 (m, 1H) 3.8 (dd, 1H), 3.95 (s, 3H) 4.85 (m, 1H) 7.2 (d, 1H) 7.55 (d, 1H) 7.7 (d, 2H), 7.8 (s, 1H) 8.1 (d, 2H)

[α]_{D}=+333.8 (c=0.985 g/cl, EtOH).

The title compound eluted at 18.57 min by HPLC as the second peak using a CHIRACEL

OD (250×4.6 mm 10 μm) column with hexane/ethanol 80/20 as the mobile phase. A 1 ml/mn flow rate was applied and 10 μL of sample prepared with the dilution of 1 mg of the title compound in 1 ml of eluent was injected. Detection of the compound was carried out with both 210 and 254 nM UV wavelengths. The other enantiomer came off at 12.8 min.

### Intermediate 23

### (2S,3S)-2,3-bis[(phenylcarbonyl)oxy]butanedioic acid-methyl 4-(1-acetyl-4-amino-2-methyl-1,2,3,4-tetrahydro-6-quinolinyl)benzoate (1:2)

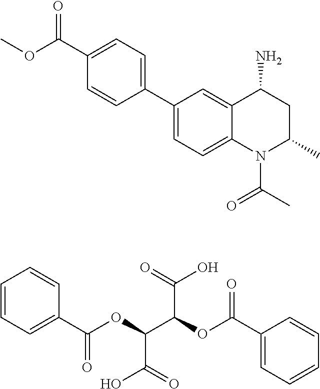

A mixture of the racemic amine Intermediate 24 (185 g,) in EtOH (600 mL) and L-(+)-lactic acid (20% in water, 450 mL) was heated to reflux during 30 minutes. After concentration under reduced pressure hexane (300 mL) was added to the residue and the resulting mixture heated to reflux 10 min. The mixture was allowed to settle and the hexane phase was discarded. The remaining paste was taken up with Et₂O (300 mL), heated to reflux during 10 minutes and allowed to settle. The Et₂O phase was discarded and the resulting paste once again was treated with hexane (200 mL), heated to reflux and allowed to settle. The hexane phase was discarded and EtOAc (2.3 L) was added to the remaining paste. The mixture was heated to reflux and allowed to stand at room temperature for 16 hours. The precipitate was filtered and washed with EtOAc (200 mL). The filtrate was made basic with addition of Na₂CO₃ and the resulting free amino was extracted with EtOAc (3×1000 mL), washed with water, dried over Na₂SO₄ and concentrated under reduced pressure. The resulting free amino (95 g) in solution in THF (950 mL) was treated with L(−)-dibenzoyltartaric acid (50.3 g, 0.14 mol) and heated to reflux 30 minutes. The resulting precipitate was allowed to stand at room temperature during 16 hours and then was filtered and washed with THF (200 ml). An HPLC monitoring of a neutralised aliquot indicated a 95.6% ee of the expected amine enantiomer. Recrystallisation of the tartaric salt in EtOH (1 L) afforded the title compound (95 g) as a single diastereomer salt. mp: 196° C.

1H NMR (300 MHz, DMSO-d6) δ ppm 0.95 (d, 3H) 1.15 (m, 1H) 2.05 (s, 3H) 2.55 (m, 1H) 3.85 (s, 3H) 4.0 (m, 1H) 4.55 (m, 1H) 5.7 (s, 1H, CH tartaric) 7.4 (m, 3H) 7.6 (m, 2H) 7.85 (m, 3H), 7.95 (m, 4H).

### Intermediate 24

### methyl 4-(1-acetyl-4-amino-2-methyl-1,2,3,4-tetrahydro-6-quinolinyl)benzoate

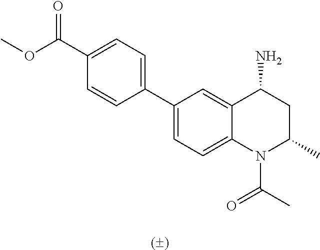

A suspension of Intermediate 25 (20.0 g) in methanol (400 mL) was refluxed, then treated with HCl 6N (18 mL). The resulting mixture was refluxed for 2 h. The suspension was filtered off on whatman and the filtrate was concentrated until dryness. Acetone (70 mL) was added to the residue, the solid was filtered off and dried. The resulting salt in ethyl acetate (300 mL) was treated with NaOH 1N (100 ml). Aqueous and organic layers were separated. Aqueous layer was extracted with CH₂Cl₂/MeOH 9:1 (300 mL). The organic layers were combined, dried and concentrated until dryness to give the title compound as a white solid (13.83 g). LCMS (Method E) Rt 2.51 MH+ 339

### Intermediate 25

### Methyl 4-[1-acetyl-4-(formylamino)-2-methyl-1,2,3,4-tetrahydro-6-quinolinyl]benzoate

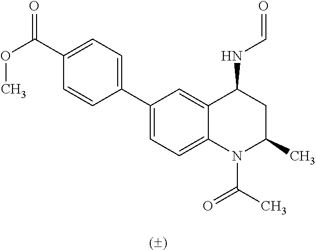

To a suspension of Intermediate 26 (1-acetyl-6-bromo-2-methyl-1,2,3,4-tetrahydro-4-quinolinyl)formamide (62.24 g) in DME (600 ml) was added palladium tetrakis (11.56 g) at room temperature. After 10 min of stirring, were added {4-[(methyloxy)carbonyl]phenyl}boronic acid (54 g) and a 2N solution of Na₂CO₃ (300 mL) and the mixture was stirred heated to reflux for 16 h. The mixture was concentrated under reduced pressure. After addition of 200 ml of DCM to the residue, the product precipitated, it was filtered and washed with water (3*100 mL). To remove the rest of the water, the solid was washed with isopropyl ether (100 ml), the solid was then added to 220 ml of warm isopropyl ether and the resulting mixture was left in the sonicator. The solid was filtered off and dried to afford the title compound as a beige solid (64.7 g)

LCMS (Method E) Rt 2.58 MH+ 367

### Intermediate 26

### [1-acetyl-6-bromo-2-methyl-1,2,3,4-tetrahydro-4-quinolinyl]formamide N-{1-methyl-7-[4-(1-piperidinylmethyl)phenyl][1,2,4]triazolo[4,3-a]quinolin-4-yl}urea

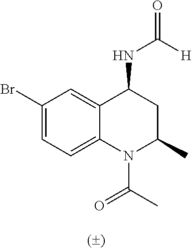

Acetyl chloride (21 mL, 0.29 mol) is added dropwise at 0° C. to a solution of Intermediate 27 (71 g, 0.26 mol) in a mixture of DCM (1 L) and pyridine (350 mL). After stirring 2 hours at 0° C. the mixture is poured into a mixture of crushed ice (2 kg) and concentrated HCl (450 mL). The product is extracted with DCM (1 L) washed with brine and dried over Na₂SO₄. Concentration under vacuo afforded the expected product as an off white solid (82 g, 100%).

1H NMR (300 MHz, CHLOROFORM-d) δ ppm 0.98 (d, 3H) 1.15 (m, 1H) 1.95 (s, 3H) 2.4 (m, 1H) 4.7 (m, 1H) 4.85 (m, 1H) 5.8 (br d, 1H) 6.85 (d, 1H) 7.15 (s, 1H) 7.25 (d, 1H) 8.2 (s, 1H)

### Intermediate 27

### (6-bromo-2-methyl-1,2,3,4-tetrahydro-4-quinolinyl)formamide

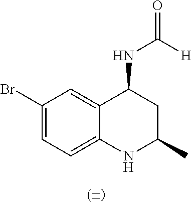

A 3 L, four neck flask under nitrogen atmosphere was charged with N-vinyl formamide (66.2 g, 0.946 mol) and dry THF (400 mL). BF₃Et₂O (239 mL, 1.9 mol) were added dropwise at −5° C. to the milky mixture. After 15 minutes Intermediate 28 (150 g, 0.473 mol) in solution in THF (1 L) was added at −5° C. After 2 h, the mixture was slowly and carefully poured in a NaHCO₃ saturated solution (5 L). Ethyl acetate (2 L) was added and the mixture was transferred to a separating funnel. The organic layer was separated and was washed 1×200 mL H₂O, 1×200 mL brine and dried (Na₂SO₄). The mixture was filtered and the solids washed 1×50 mL ethyl acetate. The filtrate was concentrated progressively until a precipitate appeared and the mixture cooled in an ice bath during 2 h. The precipitate was filtered through a Buchner funnel, and washed with 2×100 mL i-Pr₂O to deliver the title compound as a solid (71 g, 56%).

LC/MS: (Method E), m/z 269 and 271 [M+H]⁺, Rt=2.29 min;

1H NMR (300 MHz, CHLOROFORM-d) δ ppm 0.98 (d, 3H) 1.24 (q, 1H) 2.04 (ddd, 1H) 3.33 (m, 1H) 5.17 (m, 1H) 5.45 (m, 1H) 6.15 (d, 1H) 6.88 (dd, 1H) 7.00 (d, 1H) 8.11 (s, 1H)

### Intermediate 28

### [1-(1H-1,2,3-benzotriazol-1-yl)ethyl](4-bromophenyl)amine

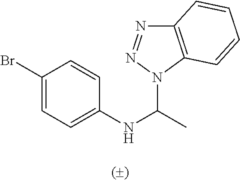

To a suspension of benzotriazole (139 g, 1.16 mol) in toluene (2 L) in a 3 L, four neck flask under nitrogen atmosphere was added at room temperature a solution of 4-bromoaniline (200 g, 1.16 mol) in toluene (300 mL). Then, via a dropping funnel was added drop wise acetaldehyde (64.7 ml, 1.17 mol) in solution in toluene (200 mL). The reaction mixture becomes progressively homogenous and then gives a precipitate. The resulting mixture is stirred 12 hours under nitrogen atmosphere and then filtered. The precipitate is recrystallised in toluene to afford the title compound as a white solid (304 g, 82%).

1H NMR (300 MHz, CHLOROFORM-d) δ ppm 2.1 (m, 3H) 4.9 (m, 0.66H) 5.15 (m, 0.33H) 6.5-6.9 (m, 3H) 7.2-8.2 (m, 7H)

### Intermediate 29

### phenyl {1-methyl-7-[4-(1-piperidinylmethyl)phenyl][1,2,4]triazolo[4,3-a]quinolin-4-yl}carbamate

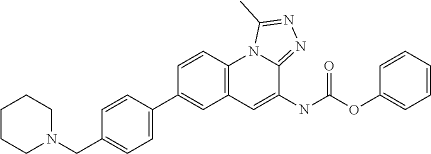

To a suspension of Intermediate 30 (3.5 g, 9.43 mmol) in anhydrous DCM (70 ml) was added pyridine (1.15 mL) followed by a dropwise addition of a solution of phenylchloroformate (1.54 mL, 12.26 mmol) in DCM (3.5 mL) and the mixture was stirred at room temperature for 3 hours. To the reaction mixture was added a saturated solution of sodium hydrogen carbonate (50 ml), and organic phase was extracted. The aqueous layer was washed with DCM. Organic layers were combined, dried over sodium sulphate and concentrated until dryness. The residue was purified by flash master with a 70 g silica cartridge eluting with CH₂Cl₂/MeOH: 95/5 to give the title compound as a yellow solid (1.49 g, 32%). LC/MS (Method D): m/z 492 (M+H)⁺, Rt: 2.88 min.

### Intermediate 30

### 1-Methyl-7-[4-(1-piperidinylmethyl)phenyl][1,2,4]triazolo[4,3-a]quinolin-4-amine

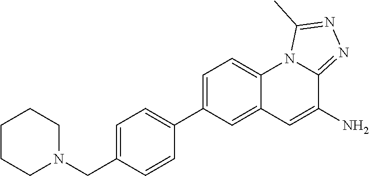

To a solution of Intermediate 31 (1.1 g, 3.97 mmol) in a mixture of toluene/ethanol (5 mL/5 mL) was added 3-(4,4,5,5-tetramethyl-1,3,2-dioxaborolan-2-yl)benzonitrile (1.18 g, 5.16 mmol), Pd(PPh₃)₄ (230 mg) and a 2N solution of Na₂CO₃ (5 mL, 10 mmol). The mixture was stirred under microwave heating and was then evaporated to dryness and diluted with dichloromethane. The organic phase was then washed with water, dried over Na₂SO₄, filtered and evaporated under reduced pressure. The residue was purified by flash column chromatography eluting with CH₂Cl₂/MeOH: 95/5 to give the title compound as a white solid (930 mg, 79%). LC/MS (Method D): m/z 300 (M+H)⁺, Rt: 2.37 min.

### Intermediate 31

### 7-Bromo-1-methyl[1,2,4]triazolo[4,3-a]quinolin-4-amine

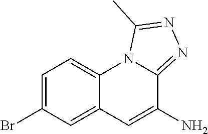

A solution of iron (16.2 g, 294 mmol) in water (110 mL) and acetic acid (1 mL) was stirred at room temperature for 15 min. Ethyl acetate (150 mL) and acetic acid (150 mL) were then added, followed by the portionwise addition of Intermediate 32 (22.3 g, 72.6 mmol). At the end of the addition, the mixture was stirred at room temperature for 24 hours. The precipitate obtained was filtered, dissolved in a mixture of AcOEt/MeOH (1/1) and then filtered. The filtrate was evaporated under reduced pressure and the residue was purified by flash column chromatography eluting with CH₂Cl₂/MeOH: 85/15 to give the title compound as a cream solid (18.78 g, 93%). LC/MS (Method D): m/z 279 (M+H)⁺, Rt: 2.39 min.

### Intermediate 32

### 7-bromo-1-methyl-4-nitro[1,2,4]triazolo[4,3-a]quinoline

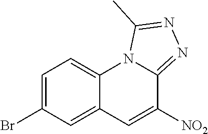

To a suspension of Intermediate 33 (111.3 g, 393 mmol) in MeOH (1.1 L) was added trimethyl orthoacetate (100 ml, 1.18 mol), a drop of H₂SO₄ and the mixture was stirred at room temperature for 4 hours. The solid was then filtered, washed with methanol, diisopropyl ether (2*300 ml) and pentane (2*300 ml). The solid was dried to give the title compound as a brown-yellow solid (117.3 g, 97%).

LC/MS (Method D): m/z 308 (M+H)⁺, Rt: 2.32 min.

### Intermediate 33

### (2Z)-6-bromo-3-nitro-2(1H)-quinolinone hydrazone

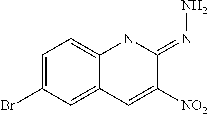

To a suspension of Intermediate 34 (122.27 g, 426 mmol) in EtOH (1.2 L) was added dropwise hydrazine hydrate (165 mL, 3.4 mol) and the mixture was stirred at room temperature for 24 hours. The reaction was not complete so hydrazine hydrate (20.63 mL, 426 mmol) was added and the mixture was stirred at room temperature for 2 hours. The solid was then filtered, washed with EtOH, diisopropyl ether (2*300 ml) and pentane (2*300 ml) and dried to give a brown-yellow solid which has some hydrazine left. The solid was stirred in water (600 ml) for 15 minutes and filtered. The solid was washed with water (2*300 ml), diisopropyl ether (2*300 ml) and pentane (2*300 ml) and dried to give the title compound as an orange solid (111.8 g, 93%)

mp: 187.4° C. LC/MS (Method D): m/z 283 (M+H)⁺, Rt: 2.76 min.

### Intermediate 34

### 6-bromo-2-chloro-3-nitroquinoline

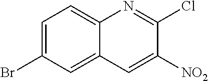

A suspension of Intermediate 35 (130 g, 483 mmol) in POCl₃ (675 mL, 7.25 mol) was stirred under reflux for 3 hours. The solution was then cooled down to 80° C., toluene (500 ml) was added slowly and the mixture was stirred at room temperature for 24 hours. The solid was washed with toluene (500 ml) and pentane (500 ml) and dried to give the title compound as yellow crystals (80.5 g, 58%).

mp: 202.8° C. LC/MS (Method D): m/z 287 (M+H)⁺, Rt: 3.36 min.

### Intermediate 35

### 6-bromo-3-nitro-2(1H)-quinolinone

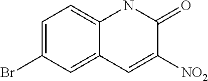

A solution of 2-amino-5-bromobenzaldehyde (140.5 g, 702.5 mmol) in EtOH (1.4 L) was added ethyl nitroacetate (156 mL, 1.4 mol) and piperidine (35 mL, 351.3 mmol) and the mixture was stirred under reflux for 18 hours. After cooling at room temperature, the solid was filtered, washed with diisopropyl ether (2*300 mL) and dried to give the title compound as a yellow solid (130.5 g, 70%).

mp: 276.5° C. LC/MS (Method D): m/z 269 (M+H)⁺, Rt: 2.42 min.

### Intermediate 36

### (3S)-3-(phenylamino)butanenitrile

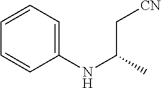

(3S)-3-aminobutanenitrile (8.6 g, 102 mmol, may be prepared as described in PCT Int. Appl., 2005100321), bromobenzene (16.16 ml, 153 mmol) and cesium carbonate (50.0 g, 153 mmol) were combined in Toluene (100 ml) under nitrogen were stirred for 45 mins. Phenylboronic acid (0.187 g, 1.534 mmol, Aldrich), palladium (II) acetate (0.188 g, 0.837 mmol, available from Aldrich) and 2-dicyclohexylphosphino-2′-(N,N-dimethylamino)biphenyl (0.443 g, 1.125 mmol, available from Aldrich) were combined in Tetrahydrofuran (THF) (6.67 ml) under nitrogen and stirred for 45 mins. The THF solution was added to the toluene solution and the reaction heated to 80° C. overnight. The reaction mixture was cooled and partitioned between EtOAc (500 ml) and water (300 ml). The aqueous layer was reextracted with EtOAc (200 ml). The combined organic layers were washed with water and brine (500 ml each) and then dried with Na2SO4, filtered and concentrated to yield an orange oil. The crude product was taken up in the minimum of DCM, applied to a 330 g Companion XL column and eluted with 5% ethyl acetate in cyclohexane for 1CV then 5-30% ethyl acetate over 12CV then held at 30% for 3CV; UV collection; 450 ml fractions. The product was isolated as an off-white solid (11.35 g).

LCMS (Method B): Rt=0.87, MH+=161

### Intermediate 37

### (3S)-3-[(4-bromophenyl)amino]butanenitrile

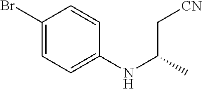

(3S)-3-(phenylamino)butanenitrile (for a preparation see Intermediate 36)(11.3526 g, 70.9 mmol) was taken up in N,N-Dimethylformamide (DMF) (200 mL) under nitrogen and cooled in an ice-bath. NBS (12.61 g, 70.9 mmol) was added and the reaction stirred. After 20 mins, the reaction was partitioned between EtOAc (1000 ml) and water (500 ml). The organic layer was washed with 2M NaOH×2, water and brine (500 ml each) and then dried with Na2SO4, filtered and concentrated to yield the product as a cream solid (17.3 g). LCMS (Method B): Rt=1.05, MH+=239

### Intermediate 38

### (3S)-3-[(4-bromophenyl)amino]butanamide

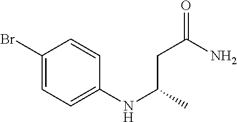

(3S)-3-[(4-bromophenyl)amino]butanenitrile (for a preparation see Intermediate 37)(17.3 g, 72.4 mmol) was taken up in Toluene (500 ml) and H2SO4 (19.28 ml, 362 mmol) added. The biphasic mixture was stirred at 60° C. After two hours, only a small amount of SM remained by LCMS so the reaction was diluted with water (500 ml) and the phases separated. The aqueous phase was basified with 10N NaOH and extracted with EtOAc(2×750 ml). The combined organics were dried with Na2SO4, filtered and concentrated to yield the product as a cream solid (17.5 g).

LCMS (Method B): Rt=0.77, MH+=257

### Intermediate 39

### 1-methylethyl {(3S)-3-[(4-bromophenyl)amino]butanoyl}carbamate

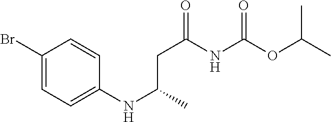

(3S)-3-[(4-bromophenyl)amino]butanamide (for a preparation see Intermediate 38, 24.9 g, 97 mmol) was taken up in Ethyl acetate (850 mL) and cooled to <−9° C.(internal). Isopropyl chloroformate (116 mL, 116 mmol, Aldrich) was added followed by slow addition of Lithium tert-butoxide (18.61 g, 232 mmol) in Tetrahydrofuran (THF) (232 mL) keeping the temperature below 0° C. The reaction was stirred for 30 mins then checked by LCMS which showed a complete reaction. The mixture was partitioned between EtOAc (1000 ml) and 2N HCl (2000 ml). The organic layer was washed with brine (2000 ml) and then dried with Na₂SO₄, filtered and concentrated to yield the product as a brown oil (17.9 g)

LCMS (Method B): Rt=1.09, MH+=343

### Alternative Method

1-methylethyl (2E)-2-butenoylcarbamate (Intermediate 42, 9.38 g, 54.8 mmol) was stirred in Toluene (281 ml) under nitrogen and (R-BINAP)ditriflatebis(acetonitrile)palladium(II) (Intermesiate 44, 3.35 g, 3.01 mmol) added. The catalyst formed a gummy ball, the solution turned to an opaque yellow mixture and was stirred for 20 mins. 4-bromoaniline (14.14 g, 82 mmol) was added, the solution turned a clear light brown and the gummy catalyst dissolved further. The mixture was stirred overnight,

Similarly a second batch of 1-methylethyl (2E)-2-butenoylcarbamate (Intermediate 42, 8.51 g, 49.7 mmol) was stirred in Toluene (255 ml) under nitrogen and (R-BINAP)ditriflatebis(acetonitrile)palladium(II) (Intermediate 44, 3.04 g, 2.73 mmol) added.

The catalyst formed a gummy ball, the solution turned to an opaque yellow mixture and was stirred for 20 mins. 4-bromoaniline (12.83 g, 74.6 mmol) was added, the solution turned a clear light brown and the gummy catalyst dissolved further. The mixture was stirred overnight.

The two reaction mixtures were combined and loaded on to a 1.5 kg Isco silica Redisep column. The column was eluted with DCM:MeOH (0%->0.5%, 19CV). The clean, product containing fractions were evaporated to a pale brown oil. The mixture was dried in a vacuum oven overnight at 40° C. to give a white solid (24.2 g, 67% overall).

LCMS (Method C): Rt=0.91, MH+=343. ee=92%.

### Intermediate 40

### 1-methylethyl[(2S,4R)-6-bromo-2-methyl-1,2,3,4-tetrahydro-4-quinolinyl]carbamate

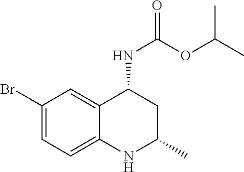

1-methylethyl {(3S)-3-[(4-bromophenyl)amino]butanoyl}carbamate (for a preparation see Intermediate 39)(17.9 g, 52.2 mmol) was taken up in Ethanol (150 mL) and cooled to below −10° C.(internal) in a CO₂/acetone bath. NaBH₄ (1.381 g, 36.5 mmol) was added followed by Magnesium Chloride hexahydrate (11.35 g, 55.8 mmol) in Water (25 mL) keeping the temperature below −5° C. The mixture was allowed to stir at <0° C. for 1 hr then warmed to r.t. and stirred for an hour. The resulting thick suspension was poured into a mixture of citric acid (25.05 g, 130 mmol), HCl (205 mL, 205 mmol) and Dichloromethane (DCM) (205 mL). The biphasic mixture was stirred at r.t. for 1 hr. LCMS showed no SM remained so the organic layer was extracted and dried with Na₂SO₄, filtered and concentrated to yield the product as a light brown solid (14.1 g).

LCMS (Method B): Rt=1.13, MH+=327

### Intermediate 41

### 1-methylethyl[(2S,4R)-1-acetyl-6-(4-formylphenyl)-2-methyl-1,2,3,4-tetrahydro-4-quinolinyl]carbamate

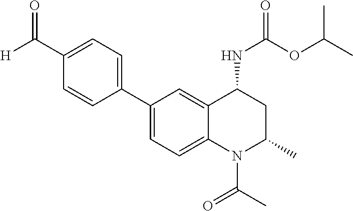

1-methylethyl[(2S,4R)-1-acetyl-6-bromo-2-methyl-1,2,3,4-tetrahydro-4-quinolinyl]carbamate (for a preparation see Intermediate 43)(1 g, 2.71 mmol), (4-formylphenyl)boronic acid (0.487 g, 3.25 mmol, available from Aldrich), Pd(Ph₃P)₄, (0.156 g, 0.135 mmol) and potassium carbonate (0.487 g, 3.52 mmol) were combined in dry Ethanol (7 ml) and dry Toluene (7.00 ml) and the reaction mixture was de-gassed for 10 mins. The reaction mixture was heated at 85° C. overnight. The reaction mixture was allowed to cool to r.t. and concentrated. The crude reaction mixture was partitioned between water (15 ml) and ethyl acetate (5 ml) and stirred at r.t. for 30 mins. A light grey solid precipitated out and was filtered off, washed with water (5 ml) and dried in a vacuum oven to give 854 mg of grey solid. LCMS (Method B): Rt=1.00, MH+=395

### Intermediate 42

### 1-methylethyl (2E)-2-butenoylcarbamate

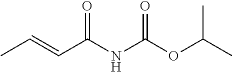

Isopropyl carbamate (30 g, 291 mmol, available from TCl) was charged to a 3 L Lara vessel and dry Tetrahydrofuran (THF) (150 ml) added. (2E)-2-butenoyl chloride (31.2 ml, 326 mmol, available from Aldrich) was added under Nitrogen and the jacket cooled to −30° C. When the solution temperature reached −17° C. 1M Lithium tert-butoxide (655 ml, 655 mmol) was added by peristaltic pump over 2 hours, keeping the reaction temperature between −10° C. and −18° C. Once the addition was complete the mixture was complete the mixture was stirred for 30 mins and brought to 0° C. Diethyl ether (450 ml) and 1M HCl (375 ml) were added and the mixture brought to 20° C. with vigourous stirring. The stirring was stopped, the layers allowed to separate and the aqueous layer run off. Brine (375 ml) was added and the mixture stirred vigourously. The stirring was stopped, the layers allowed to separate and the aqueous layer run off. The organic layer was dried (magnesium sulfate), filtered and evaporated to a brown oil (60 g). The mixture was loaded on to a 40+M Biotage silica column and eluted with DCM:ethyl acetate (1:1 to 0:1, 10CV). The product containing fractions were evaporated to dryness and loaded on to a 1500 g Redisep Isco silica column and eluted with a gradient of 0 to 40% ethyl acetate in cyclohexane. The clean, product containing fractions were evaporated to an off white solid (15.41 g). LCMS (Method C): Rt=0.68, MH+=172

### Intermediate 43

### 1-methylethyl[(2S,4R)-1-acetyl-6-bromo-2-methyl-1,2,3,4-tetrahydro-4-quinolinyl]carbamate

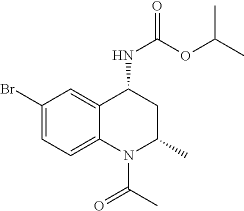

1-methylethyl[(2S,4R)-6-bromo-2-methyl-1,2,3,4-tetrahydro-4-quinolinyl]carbamate (for a preparation see Intermediate 40)(14.1 g, 43.1 mmol) was taken up in Dichloromethane (DCM) (400 mL) under nitrogen at r.t. Pyridine (10.46 mL, 129 mmol) then Acetyl chloride (4.60 mL, 64.6 mmol) were added and the reaction stirred overnight. LCMS showed a complete reaction so it was partitioned between EtOAc (2000 ml) and sat. NaHCO₃(800 ml). The organic layer was extracted and washed with water and brine (1500 ml each) and then dried with Na2SO4, filtered and concentrated to yield a purple solid. The crude product was taken up in the minimum of DCM and applied to a 330 g Companion XL column and eluted with a gradient of 12-63% Ethyl Acetate in cyclohexane. Product containing fractions were collected as an off-white solid (12.37 g).

LCMS (Method B): Rt=1.03, MH+=369

[α]_{D}=+281.1025° (T=20.7° C., 10 mm cell, c=0.508 g/100 ml, ethanol).

### Intermediate 44

### (R-BINAP)ditriflatebis(acetonitrile)palladium(II)

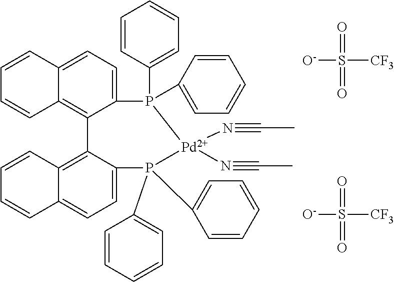

R-(+)-BINAP (6.08 g, 9.76 mmol, available from Avocado) was stirred in Dichloromethane (DCM) (626 ml) and Dichlorobis(acetonitrile)palladium (II) (2.5 g, 9.64 mmol, available from Aldrich) added. The mixture was stirred under Nitrogen for 30 mins, the suspension had not become a solution and more DCM (100 ml) was added. The mixture was stirred for a further 30 mins and Silver triflate (5.00 g, 19.47 mmol, available from Aldrich) dissolved in Acetonitrile (250 ml) was added. The mixture changed from an orange cloudy suspension to a yellow suspension. The mixture was stirred for 1 hour, filtered through celite and evaporated to an orange solid. The residue was dried under vacuum (at approximately 14 mbar) at room temperature over the weekend to give the desired product (10.69 g).

1H NMR (400 MHz, MeCN-d3) δ ppm 2.0 (s, 6H), 6.7 (d, 2H), 6.9 (br m, 4H), 7.1 (br t, 2H), 7.2 (t, 2H), 7.5-7.9 (m, 22H)

### Example 1

### 1-methylethyl((2S,4R)-1-acetyl-2-methyl-6-{4-[(methylamino)methyl]phenyl}-1,2,3,4-tetrahydro-4-quinolinyl)carbamate

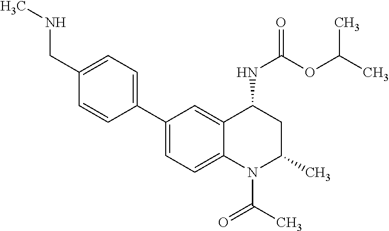

1-methylethyl[(2S,4R)-1-acetyl-6-(4-formylphenyl)-2-methyl-1,2,3,4-tetrahydro-4-quinolinyl]carbamate (for a preparation see Intermediate 41)(100 mg, 0.254 mmol) was dissolved in Methanol (3 mL) and 2M methylamine in THF (0.254 mL, 0.507 mmol) was added. The yellow solution was stirred under nitrogen at room temperature for 135 minutes at which point sodium borohydride (15.35 mg, 0.406 mmol) was added. The reaction was stirred for 1 h then left sitting overnight. The reaction was quenched with sat. aqueous sodium bicarbonate solution (1 mL) and EtOAc (8 mL) was added. A white solid was filtered off (bond elut reservoir) and found to be the desired product (34 mg). The filtrate was partitioned and the organic layer dried. Concentration of the organic layer gave 67 mg of a colourless residue which was applied to a silica 12+S Biotage column and purified eluting with a gradient of 1-5% methanolic ammonia in DCM. Concentration of the product containing fractions gave another batch of the desired product (52 mg).

LCMS (Method C): Rt 0.71, MH+=410

### Example 2

### 2-[(4S)-6-(4-Chlorophenyl)-1-methyl-8-(methyloxy)-4H-[1,2,4]triazolo[4,3-a][1,4]benzodiazepin-4-yl]-N-ethylacetamide

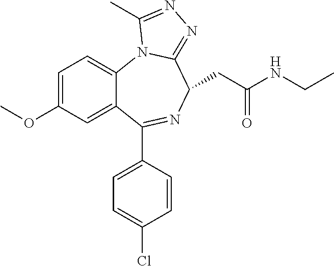

To a solution of Intermediate 1 (4.5 g, 11.4 mmol) in THF at r.t. was added DIEA (4 mL, 22.7 mmol) followed by HBTU (8.6 g, 22.7 mmol). The reaction mixture was stirred for 3 h at this temperature and ethylamine (11.3 mL, 2M in THF, 22.7 mmol) was added dropwise. The mixture was stirred overnight before being concentrated under reduced pressure. The crude material was dissolved in DCM and washed successively with water, 1N NaOH and 1N HCl. The organic layer was dried over Na₂SO₄, filtered and concentrated in vacuo. The crude solid was recrystallised in MeCN to give the title compound (4.1 g, 85% yield) as a white solid.

R_{f}=0.48 (DCM/MeOH:90/10).

Mp 140-145° C. (becomes gummy).

HRMS (M+H)⁺ calculated for C₂₂H₂₃ClN₅O₂ 424.1540. found 424.1525. Chiral HPLC: column: chiralpak AD 250×4.6 mm 10 μm; mobile phase: 60/40, EtOH/Hexane; Flow rate: 1.0 mL/min; UV wavelength: 210 and 254 nm. The title compound eluted at 5.76 min. [α]_{D}=+88.1 (c 1.0015/MeOH).

### Example 3

### 7-(3,5-dimethyl-4-isoxazolyl)-8-(methoxy)-1-[(1R)-1-(2-pyridinyl)ethyl]-1,3-dihydro-2H-imidazo[4,5-c]quinolin-2-one

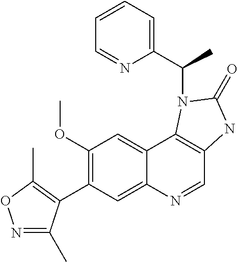

The mixture of Intermediate 9 (6 g, 18 mmol) was reacted with (1R)-1-(2-pyridinyl)ethanamine (2 eq., 4.43 g, 36 mmol) in CH₃CN (100 mL) was sirred at 110° C. for 4 hours. The reaction mixture was concentrated in vacuo. The residue was partitioned between water and DCM. The organic layer was dried over Na₂SO₄ and concentrated to dryness to give 7-(3,5-dimethyl-4-isoxazolyl)-6-(methoxy)-4-{[(1R)-1-(2-pyridinyl)ethyl]-amino}-3-quinolinecarboxamide which was used in the next step without purification. The carboxamide Intermediate (6 g) was treated with an excess of [bis(trifluoroacetoxy)iodo]benzene (19.35 g, 45 mmol) in CH₃CN (100 mL), the mixture was stirred at room temperature overnight. The reaction mixture was concentrated in vacuo, the resulting residue was dissolved in DCM and washed with water. The crude product was purified by flash chrolmatography on silica gel (DCM/MeOH, 95:5), the resulting compound was dissoled in DCM and precipitated from diethylether to give the title compound as an off-white powder (2.5 g, 33%)

LC-HRMS ES⁺ exact mass calculated for C₂₃H₂₁N₅O₃: 416.1722 (MH+). Found 416.1736, Rt=2.22 min.

LCMS (Method E) m/z 415.97(M+H)⁺, Rt=2.5 min.

### Example 4

### 7-(3,5-dimethyl-4-isoxazolyl)-8-(methoxy)-1-[(1R)-1-phenylethyl]-2-(tetrahydro-2H-pyran-4-yl)-1H-imidazo[4,5-c]quinoline

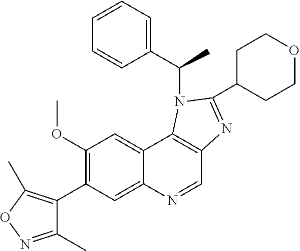

To a solution of Intermediate 15 (0.5 g, 1.28 mmol) in DCM (25 mL) at 0° C. was added tetrahydro-2H-pyran-4-carbonyl chloride (1.05 eq., 1.4 mmol, 0.15 g, Apollo Scientific), the mixture was then stirred at 0° C. for 15 minutes. The reaction mixture was hydrolysed with saturated aqueous NaHCO₃ and extracted with DCM. The organic phase was dried over Na₂SO₄ and concentrated to give the crude product which was used in the next step without purification. AcOH was added to the crude product and the mixture was stirred at 100° C. overnight. The reaction mixture was concentrated under reduce pressure, hydrolised with NaOH 1N and extracted with DCM. The organic phase was dried over over Na₂SO₄, evaporated under reduce pressure. The residue was purified by flash chromatography on silica gel (DCM/MeOH, 95:5) to give the title compound as a white powder (0.16 g, 26%).

LCMS (Method E) m/z 483 (M+H)⁺, Rt=2.93 min. [α]_{D}²⁰=−38.7° (c=0.8005 g/100 mL, CHCl₃).

¹H NMR (300 MHz, DMSO-d6, ppm) δ: 9.13 (s, 1H), 7.90 (s, 1H), 7.51-7.21 (m, 5H), 6.73 (bs, 1H), 6.51 (s, 1H), 4.09-3.85 (m, 2H), 3.69-3.45 (m, 2H), 3.31 (s, 3H), 2.27 (s, 3H), 2.07 (d, J=7 Hz, 3H), 2.06 (s, 3H), 2.04-1.92 (m, 2H), 1.91-1.80 (m, 2H).

### Example 5

### 4-{(2S,4R)-1-acetyl-4-[(4-chlorophenyl)amino]-2-methyl-1,2,3,4-tetrahydro-6-quinolinyl}benzoic acid

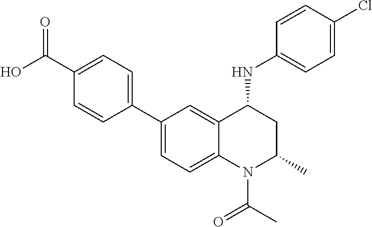

A solution of Intermediate 21 (320 mg, 0.73 mmol) in EtOH (10 ml) and 1N NaOH (1.5 ml, 1.5 mmol) was heated to reflux. After 1 hour a tlc monitoring indicated the completion of the reaction. The crude mixture was evaporated to dryness and the residue taken up in water (10 mL). Acidification of the mixture at pH=3 was carried out by addition of a 1N HCl solution. The organic materials were extracted with EtOAc (3×25 mL) and the organic phase combined and washed with brine and dried over Na₂SO₄. After concentration under vacuo the residue was taken up in a DCM/hexane mixture to give a red solid after filtration. The compound was recrystallised in EtOAC, filtered and washed with i-Pr₂O. The resulting white powder was solubilised in MeOH/H₂O, concentrated to dryness and taken up with H₂O. Finally filtration of the precipitate afforded the title compound as a white powder (147 mg), mp: 275° C.

HRMS calculated for C₂₅H₂₃N₂O₃Cl (m−H)⁻ 433.1319. found: 433.1299. Rt: 2.21 min.

1H NMR (300 MHz, CHLOROFORM-d) δ ppm 1.2 (d, 3H) 1.35 (m, 1H) 2.3 (s, 3H) 2.7 (m, 1H) 4.25 (dd, 1H) 4.95 (m, 1H) 6.65 (d, 2H) 7.15 (d, 2H) 7.25 (s, 1H) 7.55 (m, 4H), 8.15 (d, 2H)

[α]_{D}=+395 (c=0.96 g/cl, EtOH) measured at the EtOAc recrystallisation stage.

The title compound eluted at 4.51 min by HPLC as the first peak using a Chiralpak IA (250×4.6 mm 5 μm) column with tert-butyl methyl oxide (MTBE)+0.1% TFA/Ethanol:90/10 as the mobile phase. A 1 ml/mn flow rate was applied and 10 μL of sample prepared with the dilution of 1 mg of the title compound in 1 ml of eluent was injected. Detection of the compound was carried out with both 210 and 254 nM UV wavelengths. The other enantiomer came off at 5.92 min.

### Example 6

### N-{1-methyl-7-[4-(1-piperidinylmethyl)phenyl][1,2,4]triazolo[4,3-a]quinolin-4-yl}urea

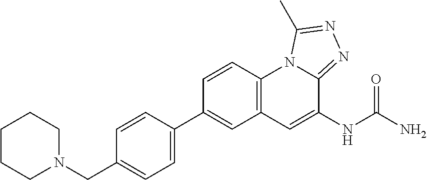

To a suspension of Intermediate 29 (0.15 g, 0.31 mmol) in ethanol (15 mL) was added a 7M solution of ammonia in MeOH (220 μL, 1.55 mmol) and the mixture was stirred at room temperature for 48 hours. The precipitate obtained was filtered, triturated with a 0.5 N solution of NaOH and washed with diisopropyl ether to give the title compound as a white solid (52 mg, 41%)

mp: >260° C. HRMS calculated for C₂₄H₂₈N₆O (M+H)⁺415.2246 found: 415.2208, Rt: 2.04 min.

### Reference Compound A

### 1,1-dimethylethyl[5-({[(4S)-6-(4-chlorophenyl)-1-methyl-8-(methyloxy)-4H-[1,2,4]triazolo[4,3-a][1,4]benzodiazepin-4-yl]acetyl}amino)pentyl]carbamate

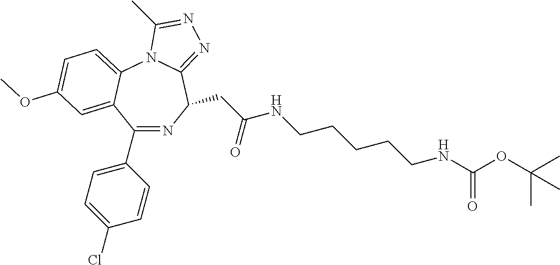

A mixture of [(4S)-6-(4-chlorophenyl)-1-methyl-8-(methyloxy)-4H-[1,2,4]triazolo[4,3-a][1,4]benzodiazepin-4-yl]acetic acid (for a preparation see Intermediate 1) (1.0 g, 2.5 mmol), HATU (1.9 g, 5 mmol) and DIPEA (0.88 ml, 5 mmol) was stirred for 80 minutes at room temperature, to this was added 1,1-dimethylethyl (4-aminobutyl)carbamate (1.05 ml, 5.0 mmol, available from Aldrich). The reaction mixture was stirred at room temperature for 2 h before it was concentrated. The residue was taken up in dichloromethane and washed with 1N HCl. The aqueous layer was extracted with dichloromethane twice. Organic layer was washed with 1N sodium hydroxide, followed by a saturated solution of sodium chloride, dried over sodium sulphate and concentrated. The residue was purified by flash-chromatography on silica using dichloromethane/methanol 95/5 to give the title compound as a yellow solid (1.2 g). LC/MS (Method D): rt=3.04 min.

### Reference Compound B

### N-(5-aminopentyl)-2-[(4S)-6-(4-chlorophenyl)-1-methyl-8-(methyloxy)-4H-[1,2,4]triazolo[4,3-a][1,4]benzodiazepin-4-yl]acetamide trifluoroacetate

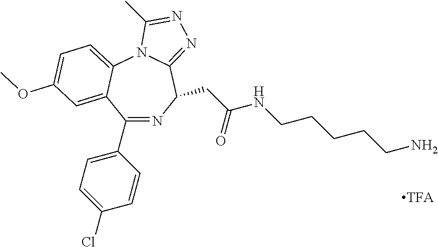

To a solution of 1,1-dimethylethyl[5-({[(4S)-6-(4-chlorophenyl)-1-methyl-8-(methyloxy)-4H-[1,2,4]triazolo[4,3-a][1,4]benzodiazepin-4-yl]acetyl}amino)pentyl]carbamate (for a preparation see Reference compound A) (0.2 g, 0.34 mmol) in dichloromethane (3 ml) was added trifluoroacetic acid (0.053 ml, 0.68 mmol) dropwise at 0° C. The reaction mixture was stirred for 3 h from 0° C. to room temperature. The reaction mixture was concentrated to dryness to afford the title compound as a hygroscopic yellow oil (200 mg)

LC/MS (Method D): rt=2.33 min.

HRMS (M+H)⁺ calculated for C₂₅H₂₉ClN₆O₂ 481.2119. found 481.2162.

### Reference Compound C

### Mixture of 5- and 6-isomers of Alexa Fluor 488-N-(5-aminopentyl)-2-[(4S)-6-(4-chlorophenyl)-1-methyl-8-(methyloxy)-4H-[1,2,4]triazolo[4,3-a][1,4]benzodiazepin-4-yl]acetamide

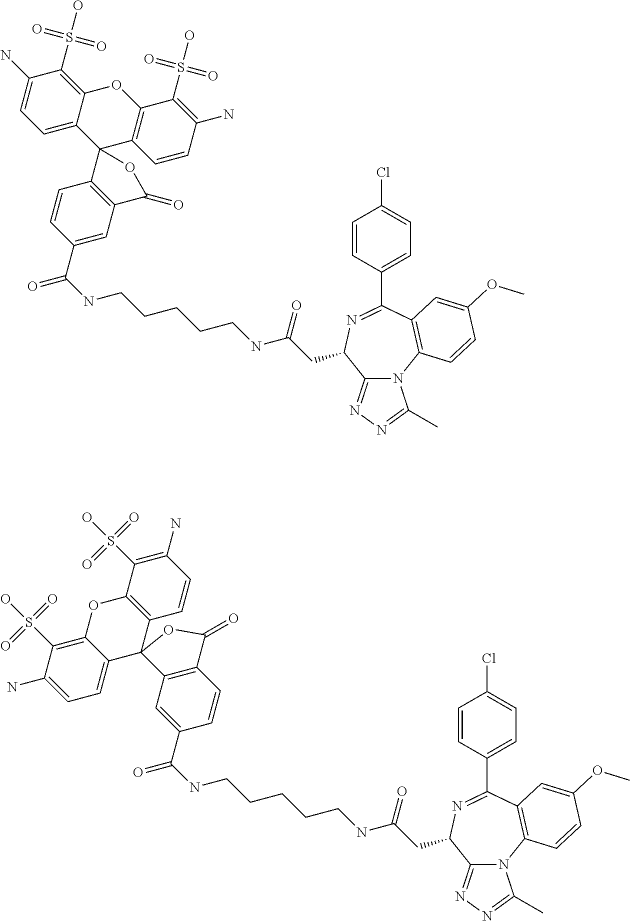

N-(5-aminopentyl)-2-[(4S)-6-(4-chlorophenyl)-1-methyl-8-(methyloxy)-4H-[1,2,4]triazolo[4,3-a][1,4]benzodiazepin-4-yl]acetamide trifluoroacetate (for a preparation see Reference compound B)(7.65 mg, 0.013 mmol) was dissolved in N,N-Dimethylformamide (DMF) (300 μl) and added to Alexa Fluor 488 carboxylic acid succinimidyl ester (5 mg, 7.77 μmol, mixture of 5 and 6 isomers, available from Invitrogen, product number A-20100) in an Eppendorf centrifuge tube. Hunig's base (7.0 μl, 0.040 mmol) was added and the mixture vortex mixed overnight. After 18 h the reaction mixture was evaporated to dryness and the residue redissolved in DMSO/water (50%, <1 ml total), applied to a preparative Phenomenex Jupiter C18 column and eluted with a gradient of 95% A: 5% B to 100% B (A=0.1% trifluoroacetic acid in water, B=0.1% TFA/90% acetonitrile/10% water) at a flow rate of 10 ml/min over 150 minutes. Impure fractions were combined and re-purified using the same system. Fractions were combined and evaporated to yield the title product (2.8 mg) as a mixture of the 2 regioisomers shown.

LC/MS (Method F): MH+=999, rt=1.88 min.

### Biological Test Methods

### Fluorescence Anisotropy Binding Assay

The binding of a compound for use in the invention to Bromodomains BRD2, BRD3 and BRD4 was assessed using a Fluorescence Anisotropy Binding Assay.

The Bromodomain protein, fluorescent ligand (Reference compound C see above) and a variable concentration of test compound are incubated together to reach thermodynamic equilibrium under conditions such that in the absence of test compound the fluorescent ligand is significantly (>50%) bound and in the presence of a sufficient concentration of a potent inhibitor the anisotropy of the unbound fluorescent ligand is measurably different from the bound value.

All data was normalized to the mean of 16 high and 16 low control wells on each plate. A four parameter curve fit of the following form was then applied:

y=a+((b−a)/(1+(10̂x/10̂c)̂d)

Where ‘a’ is the minimum, ‘b’ is the Hill slope, ‘c’ is the pIC50 and ‘d’ is the maximum.

Recombinant Human Bromodomains (BRD2 (1-473), BRD3 (1-435) and BRD4 (1-477)) were expressed in E. coli cells (in pET15b vector) with a six-His tag at the N-terminal. The His-tagged Bromodomain was extracted from E. coli cells using 0.1 mg/ml lysozyme and sonication. The Bromodomain was then purified by affinity chromatography on a HisTRAP HP column, eluting with a linear 10-500 mM Imidazole gradient, over 20 Cv. Further purification was completed by Superdex 200 prep grade size exclusion column. Purified protein was stored at −80 C in 20 mM HEPES pH 7.5 and 100 mM NaCl.

Protocol for Bromodomain BRD2: All components were dissolved in buffer composition of 50 mM HEPES pH7.4, 150 mm NaCl and 0.5 mM CHAPS with final concentrations of BRD2, 75 nM, fluorescent ligand 5 nM. 10 μl of this reaction mixture was added using a micro multidrop to wells containing 100 nl of various concentrations of test compound or DMSO vehicle (1% final) in Greiner 384 well Black low volume microtitre plate and equilibrated in dark 60 mins at room temperature. Fluorescence anisotropy was read in Envision (λex=485 nm, λEM=530 nm; Dichroic−505 nM).

Protocol for Bromodomain BRD3: All components were dissolved in buffer of composition 50 mM HEPES pH7.4, 150 mm NaCl and 0.5 mM CHAPS with final concentrations of BRD3 75 nM, fluorescent ligand 5 nM. 10 μl of this reaction mixture was added using a micro multidrop to wells containing 100 nl of various concentrations of test compound or DMSO vehicle (1% final) in Greiner 384 well Black low volume microtitre plate and equilibrated in dark 60 mins at room temperature. Fluorescence anisotropy was read in Envision (λex=485 nm, λEM=530 nm; Dichroic −505 nM).

Protocol for Bromodomain BRD4: All components were dissolved in buffer of composition 50 mM HEPES pH7.4, 150 mm NaCl and 0.5 mM CHAPS with final concentrations of BRD4 75 nM, fluorescent ligand 5 nM. 10 μl of this reaction mixture was added using a micro multidrop to wells containing 100 nl of various concentrations of test compound or DMSO vehicle (1% final) in Greiner 384 well Black low volume microtitre plate and equilibrated in dark 60 mins at room temperature. Fluorescence anisotropy was read in Envision (λex=485 nm, λEM=530 nm; Dichroic −505 nM).

Examples 1-6 were tested in the above assays and found to have a pIC₅₀≧6.0 in one or more of the BRD2, BRD2 and BRD3 assays described above.

### Lipopolysaccharide (LPS) Stimulated Whole Blood Measuring TNFα Levels Assay

Activation of monocytic cells by agonists of toll-like receptors such as bacterial lipopolysaccharide (LPS) results in production of key inflammatory mediators including TNFα. Such pathways are widely considered to be central to the pathophysiology of a range of auto-immune and inflammatory disorders.

Compounds to be tested are diluted to give a range of appropriate concentrations and 1 ul of the dilution stocks is added to wells of a 96 plate. Following addition of whole blood (130 ul) the plates are incubated at 37 degrees (5% CO2) for 30 min before the addition of 10 ul of 2.8 ug/ml lipopolysaccharides (LPS), diluted in complete RPMI 1640 (final concentration=200 ng/ml), to give a total volume of 140 ul per well. After further incubation for 24 hours at 37 degrees, 140 ul of PBS are added to each well. The plates are sealed, shaken for 10 minutes and then centrifuged (2500 rpm×10 min). 100 ul of the supernatant are removed and TNFα levels assayed by immunoassay (typically by MesoScale Discovery technology) either immediately or following storage at −20 degrees. Dose response curves for each compound was generated from the data and an IC₅₀ value was calculated.

Examples 1-6 were tested in the above assay were found to have a pIC₅₀≧6.0.

These data demonstrate that bromodomain inhibitors tested in the above assay inhibited the production of the key inflammatory mediator TNFα.

### In Vivo Mouse Endotoxemia Model

High doses of Endotoxin (bacterial lipopolysaccharide) administered to animals produce a profound shock syndrome including a strong inflammatory response, dysregulation of cardiovascular function, organ failure and ultimately mortality. This pattern of response is very similar to human sepsis and septic shock, where the body's response to a significant bacterial infection can be similarly life threatening.

To test the compounds for use in the invention groups of eight Balb/c male mice were given a lethal dose of 15 mg/kg LPS by intraperitoneal injection. Ninety minutes later, animals were dosed intravenously with vehicle (20% cyclodextrin 1% ethanol in apyrogen water) or compound (10 mg/kg). The survival of animals was monitored at 4 days.

Numbers of animals surviving at 4 days (summed across multiple repeat experiments)

| | |
|---|---|
| Vehicle | 4/66 (6%) |
| Compound of Example 3 | 55/66 (83%) |
| Compound of Example 5 | 14/24 (58%) |
| Compound of Example 2 | 24/56 (52%) |
| Compound of Example 4 | 9/24 (38%) |

These data demonstrate that bromodomain inhibitors tested in the above model gave rise to a significant animal survival effect following intravenous administration.

All publications, including but not limited to patents and patent applications, cited in this specification are herein incorporated by reference as if each individual publication were specifically and individually indicated to be incorporated by reference herein as though fully set forth.

What is claimed is:

1. A method of treating autoimmune and inflammatory diseases or conditions which comprises administering to a subject in need thereof a therapeutically effective amount of a bromodomain inhibitor.

2. A method according to claim 1 in which the bromodomain inhibitor is a compound that inhibits the binding of BET family bromodomains to acetylated lysine residues.

3. A method according to claim 2 in which the BET family bromodomain is BRD2, BRD3 or BRD4.

4. A method according to claim 1 in which the bromodomain inhibitor is a compound selected from
1-methylethyl((2S,4R)-1-acetyl-2-methyl-6-{4-[(methylamino)methyl]phenyl}-1,2,3,4-tetrahydro-4-quinolinyl)carbamate,
2-[(4S)-6-(4-Chlorophenyl)-1-methyl-8-(methyloxy)-4H-[1,2,4]triazolo[4,3-a][1,4]benzodiazepin-4-yl]-N-ethylacetamide,
7-(3,5-dimethyl-4-isoxazolyl)-8-(methoxy)-1-[(1R)-1-(2-pyridinyl)ethyl]-1,3-dihydro-2H-imidazo[4,5-c]quinolin-2-one,
7-(3,5-dimethyl-4-isoxazolyl)-8-(methoxy)-1-[(1R)-1-phenylethyl]-2-(tetrahydro-2H-pyran-4-yl)-1H-imidazo[4,5-c]quinoline,
4-{(2S,4R)-1-acetyl-4-[(4-chlorophenyl)amino]-2-methyl-1,2,3,4-tetrahydro-6-quinolinyl}benzoic acid, and
N-{1-methyl-7-[4-(1-piperidinylmethyl)phenyl][1,2,4]triazolo[4,3-a]quinolin-4-yl}urea.

5. A method according to claim 4 in which the autoimmune and inflammatory diseases or conditions involve an inflammatory response to infections with bacteria, viruses, fungi, parasites or their toxins.

6. A method according to any claim 4 in which the autoimmune and inflammatory diseases or conditions are selected from the group consisting of sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria and SIRS associated with viral infections such as influenza, herpes zoster, herpes simplex and coronavirus.

7-11. (canceled)
